(19) United States
(12) Patent Application Publication
(10) Pub. No.: US 2018/0231546 A1
(43) Pub. Date: Aug. 16, 2018

(54) Detection Of Oral Microbial Virulence Factors

(71) Applicant: The Procter & Gamble Company, Cincinnati, OH (US)

(72) Inventors: John Christian Haught, West Chester, OH (US); Sancai Xie, Liberty Township, OH (US); Benjamin Thomas Circello, Maineville, OH (US); Cheryl Sue Tansky, Forest Park, OH (US); Malgorzata Klukowska, Mason, OH (US); Thomas Glenn Huggins, Mason, OH (US); Deepa Ashok Khambe, Cincinnati, OH (US); Donald James White, Fairfield, OH (US)

(21) Appl. No.: 15/939,382

(22) Filed: Mar. 29, 2018

Related U.S. Application Data
(62) Division of application No. 15/148,663, filed on May 6, 2016, now Pat. No. 9,970,934.
(60) Provisional application No. 62/309,110, filed on Mar. 16, 2016.
(60) Provisional application No. 62/157,659, filed on May 6, 2015.

(51) Int. Cl. G01N 33/566; C12Q 1/6897; G01N 33/579

(52) U.S. Cl. CPC G01N 33/566; C12Q 2600/136; G01N 2400/50; G01N 33/579; C12Q 1/6897; G01N 2333/705

(57) ABSTRACT

Methods for detecting and quantifying toxins present in the oral cavity. The methods may include providing a biological sample, providing reporter cells expressing one or more Toll like receptors, exposing the cells to the biological sample, measuring the EC50 value of the lipopolysaccharide on activation of a Toll like receptor, quantification of the lipopolysaccharide in the biological sample.

### FIELD OF THE INVENTION

The invention relates to a method for detecting and quantifying bacterial virulence factors in oral samples, and for identifying agents for detoxifying oral virulence factors and a method for determining the effectiveness of detoxifying agents.

### BACKGROUND OF THE INVENTION

The toxins of bacterial cell walls (Henkel et al, EXS. (2010) 100: 1-29) have been associated with health related issues, such as septic shock, fever and malaise (V. Liebers et al., Am J Ind Med. (2006) 49(6): 474-91). Examples of Gram-negative cell wall toxins associated with health concerns are endotoxins, such as lipopolysaccharide (LPS), peptidogylcans, and fimbriae; and Gram-positive cell wall toxins associated with health concerns are lipoteichoic acid (LTA) and peptidoglycans. There are many other bacterial toxins, such as enterotoxins and exotoxins, as reported in Henkel et al, EXS. 2010; 100: 1-29. For the oral environment, the LPS and LTA seem to be the dominant drivers of a bacterial induced immune response, or at least the best characterized. The immune response mounted by the body in response to these toxins depends on the origin of the toxin and the exposure history of the individual to said toxin. The LPS is a component of Gram-negative bacteria that is different from strain to strain, as has been illustrated with the differences in virulence of E. coli (Raetz and Whitfield Annu. Rev. Biochem (2002) 71:635-700). LPS is composed of a lipid A fraction, core region, and may have an O-antigen. The Lipid A fraction's fatty acid composition has been shown to determine its virulence in response to its interaction with the Toll-like 4 (TLR4) receptor. The LTA has been linked to various inflammatory responses (Y. Yokoyama, et al., Acta Otolaryngol Suppl. (1996) 523:108-111) and associated with Toll-like receptor 2 (TLR2) activation. It is widely believed that only the lysed bacteria liberate LPS that can initiate an inflammatory response (CA2323630). However, Zhang et al. showed that growing bacteria secrete LPS at a level in proportion to their growth phase (H. Zhang et al. (1998) Infection & Immunity, 66(11), 5196-5201). Therefore, even a small fraction of the plaque left on the teeth after brushing could seed the inflammatory cascade due to the release of LPS from the Gram negative bacteria present in the plaque.

Methods of detecting specific microbial species have been demonstrated in the art. In US Pub. No. 2012/019735A1, methods were proposed to distinguish disease-causing bacteria via spectrophotometric methods. Though they were able to show the presence of specific microbes, their invention would not allow the user to determine the virulence level of a specific site. Further, their method requires the microbes to be cultured in the lab in order to obtain a sufficient quantity of LTA or LPS for detection. Thus, their invention lacks the ability to detect the non-culturable species present, nor would it allow for measures of toxicity of biological samples.

In U.S. Pat. No. 5,175,089, the use of the Limulus amebocyte lysate (LAL) endotoxin (LPS) assay was applied to the determination of the amount of endotoxin in the periodontal pocket. Though they were able to show overall amounts of endotoxin present, they lacked the ability to differentiate diseased versus healthy endotoxin and they were unable to quantify the level of virulence of the endotoxin. Further, their invention limited them to the Gram-negative endotoxin, as the LTA is not detectable via the LAL kit.

In US Pub. No. 2009/0047240, the chaperonin 10 (Cpn10) was used to modulate the clustering of Cpn10 in a cell line (murine RAW264) with labeled antibodies. Though they showed TLR-4, 7, and 9 reporter genes in an HEK cell line, their system would not allow for a more sensitive or low level detection needed for microbial populations with weaker activating LPS, since those genes were under the control of the NFkB binding sites only (a minimal promoter). Their system lacks the sensitivity needed to differentiate biological systems with multiple microbial species and no dominant organism present. Further, their system needs strong NFkB activators to overcome the weak promoter used in their system, thus unable to pick up weaker TLR LPS agonists, such as LPS from Porphyromonas gingivalis. Additionally, their system lacked the ability to detect TLR3 agonists, which would be deleterious to the characterization of an inflammatory disease, such as gingivitis.

US Pub. No. 2007/0160544 describes a method for determining orally deleterious bacteria. Their method calls for contacting a gingival cell with bacteria or a bacterial component and measuring an inflammatory marker. According to US Pub. No. 2007/0160544, the presence of a marker indicates inflammation and the labeling of a bacterium as deleterious. Conversely, they say that the absence of a marker indicates the bacterium is not a problem. Though they cited Toll-like receptors, which were known in the art as part of the pathway to generate cytokines, their method would have only allowed for determining the presence of a cytokine.

Since oral cells contain one or more of the receptors to which a bacterial virulence factor would activate, screening on the individual receptors requires the use of engineered cells, such as reporter cells containing the receptor gene of interest. What further complicates the use of native oral cells, such as gingival cells, is that the expression and activation of a receptor, such as a Toll-like receptor, is specific to the function of the cell. Gingival cells are less likely to respond to bacterial virulence factors, due to their constant contact with microbes in the dental plaque. Thus the need exists to have engineered cells where a direct response can be measured via a reporter system.

In addition to quantifying the virulence of microbial components and byproducts, there also exists a need for an in vitro screen of the inflammatory potential of organic and inorganic molecules, which would allow for pharmokinetic parameters to be determined.

### SUMMARY OF THE INVENTION

A method of determining bacterial virulence in an oral cavity is provided that includes providing a reporter cell expressing at least one toll-like receptor; providing a sample of oral matter; combining the sample of oral matter and the reporter cell; and measuring the toll-like receptor activation. The method may also include the additional steps of providing another sample of oral matter from an individual who used an oral care composition prior to providing another sample of oral matter; combining the another sample of oral matter and the reporter cell; measuring the toll-like receptor response; and comparing the toll-like receptor response from the sample of oral matter and another sample of oral matter.

A method for determining the virulence of lipopolysaccharide comprising providing a lipopolysaccharide; providing a reporter cell expressing at least one Toll-like receptor; combining the reporter cell with the lipopolysaccharide; measuring the toll-like receptor activation; and quantifying the lipopolysaccharide.

A method for determining the virulence of lipoteichoic acid comprising providing a lipoteichoic acid; providing a reporter cell expressing at least one Toll-like receptor; combining the reporter cell with the lipoteichoic acid; measuring the toll-like receptor activation; and quantifying the lipoteichoic acid.

The present invention includes methods of improving and/or resolving the state of gingival inflammation utilizing molecules, peptides, or proteins/enzymes that bind to, alter, or chemically modify bacterial virulence factors and/or host response mechanisms. The methods provide a means of quantifying the level of lipopolysaccharide of oral tissues; and utilizes TLR-4 and TLR-2 reporter cells line combined with the detection LPS via a fluorescence assay, such as BODIPY TR cadaverine, or endotoxin detection assay, assay to assign potency and quantification of LPS. The invention also includes assays and protocols which enable communication and demonstrations to consumers and dental professionals utilizing the TLR reporter cells.

The present invention includes methods for determining the potency of lipopolysaccharide comprising: a) providing a lipopolysaccharide sample; b) providing reporter cells expressing one or more Toll-like receptors; c) exposing the cells to the lipopolysaccharide sample; d) measuring the EC50 value of the lipopolysaccharide on activation of a Toll-like receptor; e) quantification of the lipopolysaccharide.

The present invention also includes isolating lipopolysaccharide from a growth culture of Gram negative bacteria. A lipopolysaccharide may be isolated from a biological sample. The biological sample includes, but is not limited to, an oral plaque, saliva, gingival brush samples. Toll-like receptor reporter gene assays, such as TLR4-SEAP and/or TLR2-SEAP, may be used to detect and quantify bacterial toxins, including but not limited to endotoxins, in a biological sample.

A BODIPY TR cadaverine assay may be used to detect and quantify lipopolysaccharide in a biological sample. In addition, LAL (the Limulus amebocyte lysate assay) assay or endotoxin detection assay may be used to detect and quantify the lipopolysaccharide in a biological sample.

The present invention may be directed toward a method for determining the potency of an oral biofilm comprising: a) providing a biofilm sample; b) providing reporter cells expressing one or more Toll like receptors; c) exposing the cells to the biofilm sample; d) measuring the EC50 value of the biofilm activation of a Toll like receptor; e) quantification of the lipopolysaccharide. The biofilm may be an oral plaque, including but not limited to subgingival plaque, marginal or gumline plaque, supragingival plaque.

The present invention may also be directed toward a method for determining the potency of virulence in an oral sample comprising: a) providing an oral sample; b) providing reporter cells expressing one or more Toll-like receptors; c) exposing the cells to the oral sample; d) measuring the EC50 value of the biofilm activation of a Toll like receptor; e) quantification of the lipopolysaccharide. The oral sample may include saliva, oral lavage or gingival crevicular fluid.

### BRIEF DESCRIPTION OF THE DRAWINGS

FIG. 1A is a graph showing secretion of LPS during bacterial growth in culture.

FIG. 1B is a graph showing time course of bacterial growth in culture medium.

FIG. 1C is a graph showing secretion of LPS during bacterial growth in culture.

FIG. 1D is a graph showing secretion of LPS during bacterial growth in culture at 24 hours. P. pallens and P. nigrescens released more LPS into the medium than P gingivalis.

FIG. 1E is a graph showing the amount of LPS per bacterium during bacterial growth in culture at 24 hours. P. pallens and P. nigrescens contain more LPS than P gingivalis.

FIG. 1F is a picture showing the taking of gingival plaque samples using Periopaper.

FIG. 2A is a graph showing cell culture and virulence determination of lipopolysaccharide from different bacteria.

FIG. 2B is a graph showing activation of TLR4 reporter gene in culture.

FIG. 2C is a graph showing activation of TLR4 reporter gene in culture.

FIG. 3 is a graph showing stannous fluoride inhibition of TLR4 reporter gene expression.

FIG. 4 is a graph showing stannous fluoride prevention of P. gingivalis LPS from activating TLR2 reporter gene expression.

FIG. 5 is a graph showing percentage reduction of P. gingivalis LPS on activation of TLR-4 by stannous fluoride.

FIG. 6 is a graph showing EC50 determination of subgingival plaques that activate TLR4-SEAP signal transduction. Dental plaques from unhealthy sites had a smaller EC50 than those from healthy sites, suggesting the dental plaques from unhealthy sites contain more virulence factors.

FIG. 7A is a graph showing TLR4 Evaluation of Human Subgingival Plaque (Healthy vs. Gingivitis).

FIG. 7B is a graph showing TLR4 Evaluation of Human oral lavage (Healthy vs. Gingivitis). and

FIG. 8A is a graph showing cell culture and virulence determination of LTA from different bacteria.

FIG. 8B is a graph showing cell culture and virulence determination of LPS from different bacteria.

FIG. 8C is a graph showing cell culture and virulence determination of LPS from different bacteria.

FIG. 8D is a graph showing cell culture and virulence determination of LPS from different bacteria.

FIG. 9 is an EC50 graph showing determination of subgingival plaques that activate TLR2-SEAP signal transduction.

FIG. 10A is a graph showing TLR2 Evaluation of Human Subgingival Plaque (Healthy vs. Gingivitis).

FIG. 10B is a graph showing TLR2 Evaluation of Human oral lavage (Healthy vs. Gingivitis).

FIG. 11 is a graph showing cell culture and virulence determination of flagellins from different bacteria.

FIG. 12A is a graph showing LPS Stimulated TLR Activity in Human Monocytes THP-1.

FIG. 12B is a graph showing Evaluation of Human oral lavage (Healthy vs. Gingivitis) using the IRF-Luc reporter gene in THP-1 cells.

FIG. 12C is a graph showing Evaluation of Human Subgingival Plaque (Healthy vs. Gingivitis) in THP-1 cells using the NFkB reporter gene.

FIG. 13 is a graph showing detection of LTA of different bacteria in THP1 dual reporter cells.

FIG. 14 is a graph showing clinical measurements.

FIG. 15 is graphs showing decreases in bacterial abundance in the supragingival plaques during six weeks of regimen treatment.

FIG. 16 is a graph showing citrulline concentrations in Buccal-brush samples over a six-week regimen treatment.

FIG. 16A is a figure showing the relationship between FIG. 16A-1, FIG. 16A-2, and FIG. 16A-3.

FIG. 16A-1 is graphs showing decreases in bacterial abundance in the supragingival plaques during six weeks of regimen treatment.

FIG. 16A-2 is graphs showing decreases in bacterial abundance in the supragingival plaques during six weeks of regimen treatment.

FIG. 16A-3 is graphs showing decreases in bacterial abundance in the supragingival plaques during six weeks of regimen treatment.

FIG. 17 is a graph showing protein bound ornithine in Buccal brush samples during treatment.

FIG. 18 is a diagram showing enzymes in the ornithine, citrulline and arginine cycle.

FIG. 19 is a graph showing expression of ornithine, citrulline and arginine genes during a 6-week treatment.

FIG. 20 is a graph showing citrulline concentrations increased in Buccal-brush samples in experimental gingivitis.

FIG. 21 is graphs showing protein bound citrulline in Buccal brush samples in experimentally induced gingivitis.

FIG. 22 is graphs showing concentrations of protein bound ornithine in Buccal-brush samples in experimentally induced gingivitis.

FIG. 23 is graphs showing concentrations of total ornithine in Buccal-brush samples in experimentally induced gingivitis.

FIG. 24 is graphs showing concentrations of protein bound arginine in Buccal-brush samples in experimentally induced gingivitis.

FIG. 25 is a graph showing concentrations of total arginine in Buccal-brush samples in experimentally induced gingivitis.

FIG. 26 is a graph showing citrulline inhibited cytokine production stimulated by P. gingivalis LPS 1690 in THP-1 cells.

FIG. 27 is graphs showing bacterial LTA reduces fluorescent intensity of BC in a dose dependent manner.

FIG. 28 is graphs showing LPS reduces the fluorescent intensity of BC in a dose-dependent manner.

FIG. 29 is graphs showing the supernatant of bacterial culture reduces the fluorescent intensity of BC in a dose-dependent manner.

FIG. 30 is graphs showing bacteria reduce the fluorescent intensity of BC in a dose-dependent manner.

FIG. 31 is graphs showing EC50 values.

### DETAILED DESCRIPTION OF THE INVENTION

As disclosed herein, it was surprisingly discovered that one or more methods could be used to detect and quantify the virulence in subgingival and supragingival plaques, thus distinguishing healthy gingivae from an inflamed site suffering from the symptoms of gingivitis. The state of health of the gingivae can be directly related to the level of bacterial toxins present, for example, endotoxins, and thus a reduction of these toxins, as discussed herein, in the oral cavity, as determined by screening on non-gingival engineered cells, would improve overall oral health. Additionally, the way an individual responds to the virulence factors can be quantified utilizing the individual's metabolic pathways, such as by quantifying products of the urea cycle.

Gingivitis is defined per the FDA monograph (12 CFR Part 356, Vol. 68, No. 103 (2003)) as “An inflammatory lesion of the gingiva that is most frequently caused by dental plaque. Gingivitis is characterized by tissue swelling and redness, loss of stippling (a normal state in which the surface of healthy gingiva is comprised of small lobes), glossy surface, and increased tissue temperature. The gingiva also may bleed upon gentle provocation, such as tooth brushing or may bleed spontaneously. Gingivitis is usually not painful.” Within the monograph, plaque is defined as being composed of multiple bacterial species. Those species exert a constant inflammatory pressure on the host tissues.

When the inflammation progresses to the state of gingivitis, there exists a need to quantify how severe the gingivitis is and how effective treatments from oral hygiene products are in reducing the inflammatory response. The reduction in inflammatory response due to activation of membrane bound receptors across the gingival cells is termed detoxification; and measuring the level of detoxification, which is lacking in the art, is needed to educate consumers on the efficacy of their oral hygiene.

Pathogenesis of gingivitis involves both bacteria and host responses. The present invention discloses methodologies measuring the virulence factors in the dental plaques in vitro, and also measuring effects of virulence factors on gingival tissues in vivo. These methodologies allow understanding of what virulence factor types are present in the dental plaques, and how the host responds. Importantly, the measuring of virulence factors provides a detailed assessment on the severity of gingivitis in terms of virulence factors of the microbes in dental plaques and the health status of the host. In addition, these methods help evaluate the effectiveness of a technology in preventing and treating gingivitis.

The present invention includes a methodology comprising one or more of the following steps: (1) using the endotoxin detection assay kit or BODIPY-TR cadaverine method to quantify total LPS and LTA present in a biological sample and to detect technologies that inhibit the endotoxin detection assay; (2) using the Toll like receptor assays to determine potency of purified virulence and oral dental plaques, and to measure the efficacy of technologies that neutralize toxicity of virulence factors; (3) using meta-sequencing to identify and quantitate bacteria in supragingival plaques, (4) measuring ornithine and citrulline in gingival swab samples to determine the healthy status of gingival tissue, (5) measuring protein and mRNA levels to determine the levels of host responses, and determining citrulline activities in inhibiting LPS-induced production of proinflammatory cytokines.

The above-mentioned methods and chemistry may be applied in a strip form to the outer surface of the tooth and gumline. The strip may contain color or fluorescence reagents to interact with the virulence factors present and thus allow for semi-quantitative determination of the virulence present.

This would allow for rapid assessment of the level of severity of gingivitis and/or periodontal disease or to ascertain the effectiveness of oral products. This execution of the described methods allows for consumers to ascertain the state of their dental health at home or allows for professionals, such as dentists, to rapidly measure the state of a patient's oral health.

The methods described above can be used to determine the cellular impact of organic and inorganic molecules, as long as there is an interaction between the molecule and the targeted receptor. Further, the methods could be used to ascertain if a molecule would cause an irritation or inflammatory response. The receptor in question would be used in a reporter system, as described herein, and the molecular impact of the molecule in question determined. The EC50 value of the molecule in question would be determined using the receptor associated with the biological response, thus reducing or eliminating the need to do animal testing.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. (i.e. room temperature) unless otherwise specified

As used herein, the word “include,” and its variants, are intended to be non-limiting, such that recitation of items in a list is not to the exclusion of other like items that may also be useful in the materials, compositions, devices, and methods of this invention.

As used herein, the word “or” when used as a connector of two or more elements is meant to include the elements individually and in combination; for example X or Y, means X or Y or both.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include hair coloring compositions, oral care compositions, after shave gels and creams, pre-shave preparations, shaving gels, creams, or foams, moisturizers and lotions, cough and cold compositions, leave-on skin lotions and creams, shampoos, conditioners, shower gels, bar soaps, toilet bars, antiperspirants, deodorants, depilatories, lipsticks, foundations, mascara, sunless tanners and sunscreen lotions.

By “oral care composition”, as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice”, as used herein, includes tooth or subgingival-paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “teeth”, as used herein, refers to natural teeth as well as artificial teeth or dental prosthesis.

Virulence factors are molecules produced by pathogenic microbes that contribute to the pathogenicity of the organism and enable them to invade and proliferate in the host, and evade host immune surveillance. Virulence factors include, but are not limited to the following: Gram positive and Gram negative cell wall components, such as lipopolysaccharide and lipoteichoic acids, bacterial DNA, flagellin, peptidoglycan, adhesins, invasins, and antiphagocytic factors, hemolysins, bacterial metabolites, fimbriae, outer membrane vesicles, bacterial proteins or bacterial enzymes. A reduction in virulence or “detoxification”, based on reduced activation of Toll-like receptors, can be used to measure the effectiveness of various treatments.

The term “detoxification” or “detoxify” or “detox” as used herein, refers to the neutralization, reduction, or removal of microbial virulence factors as measured by a reduction in the activation of a receptor known to be responsive with a virulence factor from an engineered cell, such as a reporter cell line. In certain embodiments a determination of virulence is assigned based on the activation of one or more of the Toll-like receptors.

The present invention includes obtaining a sample of oral matter. The oral matter can include gum-line plaque, subgingival plaque, supragingival plaque, interstitial plaque, gingival crevicular fluid (GCF), gingival biopsy, saliva, or tongue swab. The oral matter may be obtained by any method known in the art, for example, subgingival plaque sample may be collected physically by scraping or by using paper points. The plaque may be collected off of the tooth beneath gums from the sulcus, the developed periodontal pocket, or at the gumline. For example, each paper point can be placed in the pocket between the tooth and the gingiva for 10 seconds. After 10 seconds, a paper point can be removed and placed into a pre-labeled 1.5 ml tube with 700 μl phosphate-buffered saline. The sampling procedure can be repeated with three more paper points. After all four paper points are collected, the 1.5 ml tube will be closed, vortexed for 30 seconds and placed on dry ice until the samples are stored in a −80° C. freezer. Other methods of collection could include a mechanical device to help release the plaque from the tooth surface, such as a sonic descaler. The oral matter may be obtained both before and after treatment of an oral site from which the oral matter is obtained. An oral site from which the oral matter is obtained includes host tissues and bacterial matters. Further treatment of an oral site may be more than once and may include multiple different treatments, for example a regimen, such as brushing teeth followed by mouthrinse. In addition to obtaining oral matter before and after completed treatments, oral matter may be obtained between separate treatments, for example between the brushing of teeth followed by the use of mouth rinse.

The sample of oral matter is combined with a Toll-like receptor in a reporter cell. Examples of Toll-like Receptors that can be used in the present invention include TLR 2, TLR 4, TLR 5, and TLR 9. Human TLR cDNA (TLR 1, 2, 3, 4, and 5) was first cloned in 1998 and their sequences were published (Rock F L, Hardiman G, Timans J C, Kastelein R A, Bazan J F. A family of human receptors structurally related to Drosophila Toll. Proc Natl Acad Sci USA. 1998 Jan. 20; 95:588-93). Rock et al. discovered that cytoplasmic domains of Drosophila Toll and human interleukin 1 receptors shared high sequence homology, and hypothesized that both molecules trigger related signaling pathways tied to the nuclear translocation of Rel-type transcription factors. They cloned a class of putative human receptors with a protein architecture that is similar to Drosophila Toll in both intra- and extracellular segments. They hypothesized that the five human Toll-like receptors—named TLRs 1-5—are probably the direct homologs of the fly molecule and, as such, could constitute an important and unrecognized component of innate immunity in humans. Soon after the DNA sequences for TLR1 to 5 were published, other TLR DNA sequences were also revealed. For example, TLR6 cDNA sequence was reported in 1999 (Takeuchi O1, Kawai T, Sanjo H, Copeland N G, Gilbert D J, Jenkins N A, Takeda K, Akira S. A novel member of an expanding toll-like receptor family. Gene. 1999 Apr. 29; 231(1-2):59-65). cDNA sequences of human TLR7, TLR8 and TLR9 were reported in 2000 (Chuang TH1, Ulevitch R J. Cloning and characterization of a sub-family of human toll-like receptors: hTLR7, hTLR8 and hTLR9. Eur Cytokine Netw. 2000 September; 11(3):372-8).

| SEQ | |
|---|---|
| ID | |
| NO | Sequence |
| 1 | Human Toll-Like Receptor 1 Amino Acid sequence |
| 2 | Human Toll-Like Receptor 2 Amino Acid sequence |
| 3 | Human Toll-Like Receptor 3 Amino Acid sequence |
| 4 | Human Toll-Like Receptor 4 Amino Acid sequence |
| 5 | Human Toll-Like Receptor 5 Amino Acid sequence |
| 6 | Human Toll-Like Receptor 6 Amino Acid sequence |
| 7 | Human Toll-Like Receptor 7 Amino Acid sequence |
| 8 | Human Toll-Like Receptor 8 Amino Acid sequence |
| 9 | Human Toll-Like Receptor 9 Amino Acid Sequence |

A sequence listing that sets forth the amino acid sequences for SEQ ID NO: 1 to 9 herein is being filed concurrently with the present application as an ASCII text file titled “13837M_AA_Sequence_Listing_ST25.” The ASCII text file was created on 29 Mar. 2016 and is 68 Kbytes in size. In accordance with MPEP § 605.08 and 37 CFR § 1.52(e), the subject matter in the ASCII text file is incorporated herein by reference.

TLR proteins can form heterodimers or homodimers. There are 10 TLR genes identified in humans. Their gene products form homodimers or heterodimers in cell membranes. For example, TLR1 can form heterodimers with TLR. Similarly, TLR6 also can assemble a heterodimer with TLR2. On other hand, TLR4 forms a homodimer. Toll-like receptors (TLRs) are a class of proteins that play a key role in the innate immune system as well as the digestive system. They are membrane-spanning, non-catalytic receptors usually expressed in cells such as macrophages, dendrite cells, and gingival epithelial cells that recognize structurally conserved molecules derived from microbes. Once these microbes have breached physical barriers such as the skin or intestinal tract mucosa or oral epithelial cells they are recognized by TLRs, which activate immune cell responses. Toll-like receptors were targeted because they are the primary sensors of virulence factors produced by the microbes in the dental plaques. TLR 1, 2, 4, 5 and 6 are expressed in the cell plasma membranes, providing easy access for the virulence factors to be sensed by the host receptors. TLR 3, 7, 8, and 9 are located on the membranes of endosomes; and as virulence factors are phagocytosed into the cells, they also gain access to TLR 3, 7, 8 and 9

A reporter cell refers to a eukaryotic cell, such as, but not limited to, HEK 293T, human monocyte (THP1), Chinese hamster ovary (CHO) cell, murine cells, or monkey kidney epithelial (Vero) cells engineered to express a predetermined number of TLR receptors, for example a single TLR receptor; which is in contrast to gingival cells that express multiple functional TLR receptors. Thus, one type of engineered reporter cells respond to only one type of virulence factors in the dental plaques. In contrast, gingival cells express several types of functional TLRs, and can't be used to identify a single type of virulence factor in dental plaques. The output from gingival cells is the sum of various virulence factors in the dental plaques. HEK 293T cells can be used as reporter cells, as they are easy to maintain and have similar gene expression profiles to oral epithelial cells, making them a closer match to the gene expression of gingival cells, such that the results will mirror in vivo results. In contrast to naturally occurring gingival cells the reporter cells of the present invention are easy to maintain in the lab, and stable in phenotypes. Further, the reporter cells make detection of virulence factors simpler, are more reproducible, increase accuracy, provide higher throughput are more specific and more quantifiable.

Individual TLR receptor genes are stably transfected to HEK 293 cells as described by Invivogen (/PDF/HEK_Blue_htlr4_TDS.pdf). HEK-Blue™ hTLR4 Cells are designed for studying the stimulation of human TLR4 (hTLR4) by monitoring the activation of NF-kB. HEK-Blue™ hTLR4 Cells were obtained by co-transfection of the hTLR4 gene, the MD-2/CD14 co-receptor genes and a secreted embryonic alkaline phosphatase (SEAP) reporter gene into HEK293 cells. The SEAP reporter gene is placed under the control of an IL-12 p40 minimal promoter fused to five NF-kB and AP-1-binding sites (Supplement 1. HEK-Blue™ hTLR4 Cells SEAP Reporter 293 cells expressing the human TLR4 gene, Catalog # hkb-htlr4, Version #15C04-MM (/PDF/HEK_Blue_htlr4_TDS.pdf). As compared to measuring an immune response the reporter genes of the present invention allow rapid, specific and reproducible measurements of virulence factors.

The level of receptor activation can be determined by any method known in the art for the type of reporter gene used. For example if an NFkB-SEAP reporter gene is used, one could measure the production of SEAP in the culture medium. The reporter cells can be treated with virulence factors, or dental plaque matters collected before or after treatments. Expression of the reporter gene will be stimulated and SEAP secreted into the medium when stimulated by virulence factors. The level of reporter gene product SEAP can be readily measured with commercial kits, and will be proportional to the amount of particular types of virulence factors. Similarly, if an NFkB-luciferase, NFkB-beta-lactamase, or other reporter genes are used, available kits can be used to measure the reporter gene products.

The potency can then be determined based on parameters such as, EC50 and fold of stimulation. EC50 provides a measurement on the amount of virulence factors needed to mount an inflammatory response, and the fold of stimulation is indicative of the severity of inflammatory responses the virulence factors cause. The EC50 is used to determine potency; wherein “potency” as defined by the Merck Manual, refers to the concentration (EC50) of a chemistry required to produce 50% of the chemistry's maximal effect as depicted by a graded dose-response curve. EC50 equals Kd (Dissociation constant, which is a measure of 50% of the substance in question bound to the receptor) when there is a linear relationship between occupancy and response. Often, signal amplification occurs between receptor occupancy and response, which results in the EC50 for response being much less (ie, positioned to the left on the abscissa of the log dose-response curve) than Kd for receptor occupancy. Potency depends on both the affinity of a compound for its receptor, and the efficiency with which a compound-receptor interaction is coupled to response. The dose of a compound required to produce an effect is inversely related to potency. In general, low potency is important only if it results in a need to administer a compound in large doses that are impractical. Quantal dose-response curves provide information on the potency of a compound that is different from the information derived from graded dose-response curves. In a quantal dose-response relationship, the EC50 is the dose at which 50% of individuals exhibit the specified quantal effect.

In the present invention, the activities of endotoxins or lipopolysaccharides can be measured using an endotoxin detection assay, or LAL assay. The Limulus Amebocyte Lysate (LAL) test has been used to detect LPS. LAL is derived from the blood cells, or amebocytes, of the horseshoe crab, Limulus polyphemus. At present, some main endotoxin detection agents are derived from recombinant proteins. Thereinafter, endotoxin detection assay and LAL assay are used interchangeably.
A. A method of determining bacterial virulence in an oral cavity comprising:
   a. providing a reporter cell expressing at least one toll-like receptor;
   b. providing a sample of oral matter;
   c. combining the sample of oral matter and the reporter cell;
   d. measuring the toll-like receptor activation.
B. The method of paragraph A, wherein the toll-like receptor is at least one of TLR1 to TLR9.
C. The method according to paragraph A or B, wherein the reporter cell expresses at least of one TLR2 or TLR4.
D. The method according to any one of paragraphs A to C, wherein the toll-like receptor activates a reporter gene, preferably wherein the reported gene is a secreted embryonic alkaline phosphatase (SEAP) reporter gene.
E. The method according to any one of paragraphs A to D, wherein the sample of oral matter is at least one of gum-line plaque, subgingival plaque, supragingival plaque, interstitial plaque, gingival crevicular fluid (GCF), gingival biopsy, saliva, or tongue swab, preferably wherein the sample of oral matter comprises virulence factor that is at least one of Gram positive cell wall components, Gram negative cell wall components, bacterial DNA, flagellin, peptidoglycan, bacterial metabolites, fimbriae; outer membrane vesicles bacterial proteins, or bacterial enzymes.
F. The method of paragraph E, wherein the sample of oral matter comprises virulence factor that is at least one of lipopolysaccharide or lipoteichoic acid.
G. The method according to any of paragraphs A to F comprising the additional steps of:
   e. providing another sample of oral matter from an individual who used an oral care composition prior to providing another sample of oral matter;
   f. combining the another sample of oral matter and the reporter cell;
   g. measuring the toll-like receptor response;
   h. comparing the toll-like receptor response from the sample of oral matter and another sample of oral matter.
H. The method of paragraph G, where the sample of oral matter and the another sample of oral matter are from the same individual.
I. A method for determining the virulence of lipopolysaccharide comprising:
   a. providing a lipopolysaccharide;
   b. providing a reporter cell expressing at least one Toll-like receptor;
   c. combining the reporter cell with the lipopolysaccharide;
   d. measuring the toll-like receptor activation;
   e. quantifying the lipopolysaccharide.
J. The method of paragraph I, wherein the Toll-like receptor activation is determined by measuring at least one of EC50 or fold of stimulation.
K. The method according to paragraph I or J, wherein the Toll-like receptor is at least one of TLR1 to TLR9.
L. The method according to any of paragraphs I to K, wherein the reporter cell expresses at least one of TLR2 or TLR4.
M. The method according to any of paragraphs I to L, wherein the Toll-like receptor activates a reporter gene.
N. The method according to paragraph M, wherein the reported gene is a secreted embryonic alkaline phosphatase (SEAP) reporter gene.
O. The method according to any of paragraphs I to N, wherein the lipopolysaccharide is quantified using at least one of limulus amebocyte lysate assay or BODIPY TR cadaverine assay.

### EXAMPLES

### Example 1—Determination of Secreted Lipopolysaccharide by Bacteria

Growth of bacteria: A 1 ml aliquot of a 24 hour culture of E. coli ATCC 8739 was used to inoculate 100 ml of Luria-Bertani (LB) broth in a 250 ml baffled flask. This culture was then incubated at 37° C. with agitation (220 rpm) and sampled at 30 minute intervals. Samples were assessed for turbidity (OD₆₀₀) in a SpectraMax platereader M3 (Molecular Devices, Sunnydale, Calif.), which is one method of monitoring the growth and physiological state of microorganisms. The sample turbidity was then recorded and the samples were centrifuged at 5000 RPM for 10 min at room temperature. The supernatant, thereinafter referred to as “supernatant of bacterial culture”, was subsequently analyzed for LPS content using the procedure as described below.

Twenty ml aliquots of MTGE broth (Anaerobe Systems, Morgan Hill, Calif.) were inoculated with P. gingivalis ATCC 33277, P. pallens ATCC 700821, or P. nigrescens ATCC 25261. These cultures were incubated overnight in a Whitely A45 Anaerobic Workstation (Don Whitley Scientific, Frederick, Md.) at 37° C. with an 85:10:5 N₂:CO₂:H₂ gas ratio. One ml aliquots of these starter cultures were then used to inoculate 99 ml of membrane-Tryptone Glucose Extract (m-TGE) broth in a 250 ml baffled flask. These cultures were then incubated under agitation (200 rpm) as previously described and sampled at regular intervals. Samples were assessed for turbidity (OD₆₀₀) in a Tecan Infinite m200 Pro (Tecan Trading AG, Switzerland) and then centrifuged at 16,100×g for 10 min at room temperature. Supernatants were decanted and passed through a 0.22 μM filter prior to analysis for LPS content.

In the experiment, only OD600 was measured. For the sake of consistency in following experiments, we converted OD600 readings into bacterial numbers, even though the relationship between OD600 readings and bacterial numbers is varied for each bacterium. The number of bacteria was estimated based on spectrophotometer readings at OD₆₀₀ (OD₆₀₀ of 1.0=8×10⁸ cells/ml).

The Limulus Amebocyte Lysate Assay (LAL) is an assay to determine the total amount of lipopolysaccharide (LPS) in the sample tested (Pierce LAL Chromogenic Endotoxin Quantitation Kit, ThermoFischer Scientific, Waltham, Mass.). The assay was performed following manufacturer's instruction. Ninety-six-well microplates were first equilibrated in a heating block for 10 min at 37° C. Fifty μl each of standard or sample was dispensed into the microplate wells and incubated with plate covered for 5 min at 37° C. Then 50 μl LAL was added to each well. Plates were shaken gently and incubated for 10 min at 37° C. 100 μl of chromogenic substrate was added and incubated for 6 min at 37° C. Finally, 50 μl Stop Reagent was added and the absorbance was measured at 405-410 nm on Spectramax M3 platereader (Molecular Device, Sunnyvale, Calif.).

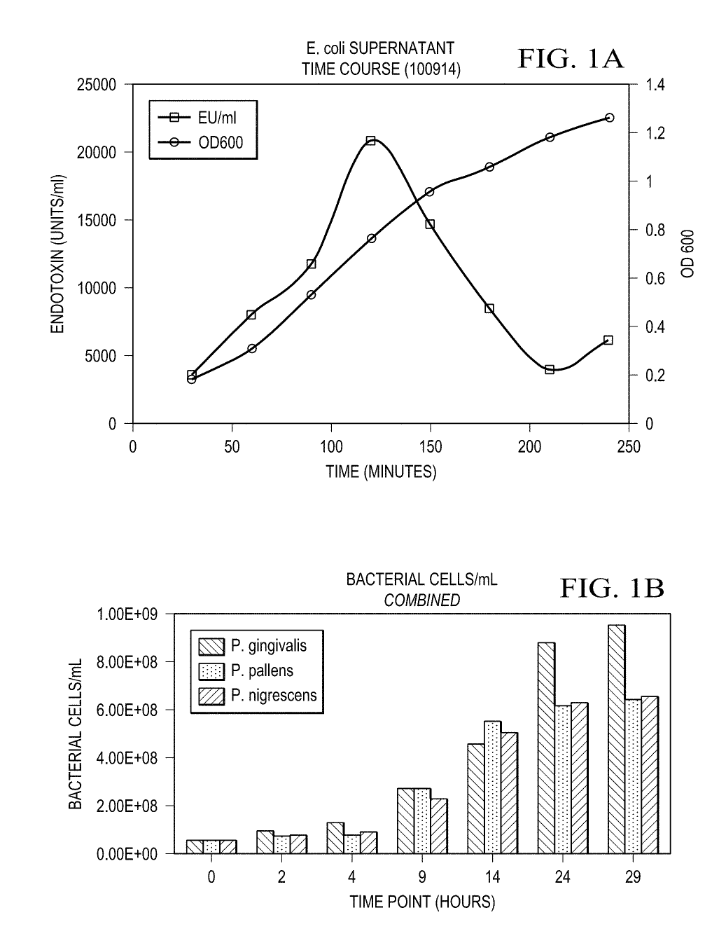

FIGS. 1A, 1C, and 1D show the ability of microbes to shed LPS as part of their normal growth cycle. This data shows the need to deliver chemistry to the subgingival plaque to effectively mitigate the LPS, since tooth brushing generally does not remove the subgingival plaque.

The LPS, as measured by the LAL kit reported in endotoxin unit per ml (EU/ml), was shed by the bacteria (E. coli K12) as depicted in FIG. 1A. The growth media began to be depleted of complex sugars around 120 minutes, as reflected in the bacterial growth curve in FIG. 1B, where the LPS shedding started to decline. This data gave a reason to believe that a mature biofilm/plaque could supply a constant level of LPS to the host cells, if food sources were present. The LPS would then have the ability to induce an inflammatory response from the host cells.

Importantly, LPS are secreted into the supernatant of bacterial culture (FIG. 1D). LPS also exists in bacterial walls (FIG. 1E). Again, this data further enforce the need to deliver chemistry to the subgingival plaque to effectively mitigate the LPS, since tooth brushing generally does not remove the subgingival plaque.

### Example 2—LAL Assay to Quantify Virulence Factors in Dental Plaques

Seven panelists, with at least three bleeding sites, took part in the testing. A licensed dental hygienist collected subgingival plaque samples. Samples were taken at the tooth/gum interface (buccal surfaces only) using care to avoid contact with the oral soft tissues. Six subgingival plaque sites were sampled from each panelist (3 healthy and 3 unhealthy sites). Unhealthy teeth had bleeding sites with pockets greater than 3 mm and healthy sites had no bleeding with pocket depth less than 2 mm Prior to sampling, panelists were instructed to abstain for 12 hours from oral hygiene and refrain from eating, chewing gum, drinking (except small sips of water). Next, panelists had their marginal plaque collected with a curette at the sampling sites. Then, from the same site, subgingival plaque samples were collected with 3 consecutive paper points as shown in FIG. 1F. The sampling sites were isolated with cotton rolls and gently air-dried. Paper points (PROFLOW incorporated, Amityville, N.Y.) were gently placed for 10 seconds into the pocket until a minimum of resistance was felt. After 10 seconds, paper points were removed and placed into pre-labeled 1.5 ml tubes. The same sampling procedure was repeated with 2 more paper points (paper points go into separate tubes). The first, second and third sample paper points from a healthy site of all panelists were pooled separately into three tubes, labeled as paper point 1, 2 and 3, respectively. Similarly the unhealthy site samples were also pooled.

TABLE 1 showed that unhealthy dental plaques contained more endotoxins than the healthy dental plaques. One m1 PBS was added to each pooled sample in the 1.5 ml tube. Bacteria were lysed in a MolBio Fast Prep bead beater (MP Biomedicals, Santa Ana, Calif.). Samples were centrifuged for 10 min at 10,000 RPM at 4° C., supernatants were collected and analyzed with LAL assay kits following manufacturer's instruction as described in EXAMPLE 1.

TABLE 1
Protein concentrations and endotoxin
levels in the pooled dental plaque samples.

| | Endotoxin |
|---|---|
| Dental plaque | (endotoxin unit) |
| Healthy paperpoint 1 sub plaque | 1284 |
| Healthy paperpoint 2 sub plaque | 476 |
| Healthy paperpoint 3 sub plaque | 361 |
| Healthy Marginal Plaque | 23180 |
| Unhealthy paperpoint 1 sub plaque | 3371 |
| Unhealthy paperpoint 2 sub plaque | 1732 |
| Unhealthy paperpoint 3 sub plaque | 16111 |
| Unhealthy Marginal Plaque | 80277 |

It was expected that the marginal plaques in unhealthy sites had more endotoxins than those in the healthy sites (TABLE1) within the same subjects. Three samples were taken from subgingival pockets with three paper points sequentially, named paper point 1, 2 and 3. Again, the subgingival plaques taken by the paper point 1 had more endotoxins in the unhealthy sites than in the healthy sites (TABLE 1). The same is true for the samples taken by paper point 2 and 3. Importantly, dental plaques in the unhealthy subgingival pockets possessed more endotoxins than plaques from healthy pockets. This may explain why unhealthy gingiva are prone to bleeding upon probing.

### Example 3—Technology Development Using the LAL Assay Kit

The LAL assay, as described in EXAMPLE 1, was modified for development of technology which inhibits LPS from activating a proenzyme in the LAL assay. The Thermo Scientific Pierce LAL Chromogenic Endotoxin Quantitation Kit is a quantitative endpoint assay for the detection of LPS, which catalyzes the activation of a proenzyme in the modified Limulus Amebocyte Lysate (LAL). The activated proenzyme then splits p-Nitroaniline (pNA) from the colorless substrate, Ac-Ile-Glu-Ala-Arg-pNA. The product pNA is photometrically measured at 405-410 nm. If SnF2 binds to LPS, the latter can't react with the proenzyme in the LAL kit. Consequently, the proenzyme is not activated, and the colorless substrate Ac-Ile-Glu-Ala-Arg-pNA will not split and no color product is produced. P. gingivalis LPS 1690 (1 ng/ml), or E. coli LPS (1 ng/ml), and stannous fluoride and other materials (50 and 500 μM), as listed in TABLE 2, were dissolved in endotoxin-free water. Then 50 μl LAL was added to each well. Plates were shaken gently and incubated for 10 min at 37° C. 100 μl of chromogenic substrate was added and incubated for 6 min at 37° C. Finally, 50 μl Stop Reagent was added and the absorbance was measured at 405-410 nm on Spectramax M3 plate reader (Molecular Device, Sunnyvale, Calif.).

As shown in TABLE 2, SnF2 and some other compounds inhibited LPS activities in LAL assays

TABLE 2
Inhibition of LPS activities on LAL Assays

| | Inhibition of LAL activity % | | | |
|---|---|---|---|---|
| | P. gingivalis LPS | | E. coli LPS | |
| | 1690 1 ng/ml | | 1 ng/ml | |
| Samples | 500 uM | 50 uM | 500 uM | 50 uM |
| Tin (II) fluoride | 60 | 49 | 92 | 87 |
| stannous chloride | 48 | 21 | 89 | 65 |
| Cetylpyridinium chloride | 103 | 77 | 103 | 46 |
| monohydrate | | | | |
| Chlorhexidine | 102 | 38 | 97 | 57 |
| zinc citrate, dihydrate | 104 | 57 | 104 | 82 |
| zinc lactate | 58 | 0 | 66 | 0 |
| potassium oxalate | 8 | 0 | 1 | 6 |
| Triclosan (irgasan) | 0 | 0 | 10 | 0 |
| 1-Hydroxypyridine-2-thone | 0 | 0 | 2 | 6 |
| zinc salt | | | | |
| sodium fluoride | 0 | 0 | 4 | 5 |
| Carboxymethyl cellulose | 0 | 0 | 2 | 0 |
| sodium | | | | |

### Example 4—Binding of BODIPY-TR-Cadaverine to LPS and Displacement of LPS Binding by Stannous Fluoride

In addition to LAL quantification of LPS, the BODIPY method can be utilized to assess the level of LPS. Detoxifying technologies are able to target and neutralize bacterial virulence factors, such as LPS and LTA. To develop such LPS and LTA sequestration technologies, a high throughput screening was employed to identify molecules that disrupt the activation of a Toll-like receptor by LPS and LTA, and other virulence factors. The high throughput screen utilizes the fluorescent dye BODIPY-TR-cadaverine 5-(((4-(4,4-difluoro-5-(2-thienyl)-4-bora-3a,4a-diaza-s-indacene-3-yl)phenoxy)acetyl)amino)pentylamine hydrochloride (BC fluorescent dye), purchased from Life Technologies (Carlsbad, Calif.), as described previously by Wood, Miller and David (2004) (Comb Chem High Throughput Screen 2004 May; 7(3):239-49. Anti-endotoxin agents. 1. Development of a fluorescent probe displacement method optimized for the rapid identification of lipopolysaccharide-binding agents.). The experiment was carried out at 22° C. E. coli LPS (15 μg/ml) was mixed in a well of a 96-well solid black flat bottom plate (Corning Incorporated, Corning, N.Y.) with SNF2 and cetylpyridinium chloride in 30 μl of 50 mM Tris buffer at pH 7.4 for 10 mM, and then 20 μl of 60 μM BC fluorescent dye was added right before fluorescence measurement at 22° C., or room temperature. Fluorescence measurements were performed in a SpectraMax M3 automated 96 well plate reader (Molecular Device, Sunnyvale, Calif.). The excitation wavelength was 580 nM and the fluorescence emission was measured at 620 nM.

As shown in TABLE 3, leading anti-gingivitis technologies, such as stannous fluoride, displaced LPS from BODIPY TR cadaverine.

TABLE 3
Displacement of LPS from binding to BODIPY TR cadaverine

| Concentrations | Displacement % | |
|---|---|---|
| mM | Stannous fluoride | Cetylpyridinium chloride |
| 0.16 | 2.45 | 9.98 |
| 0.31 | 2.79 | 31.11 |
| 0.63 | 12.69 | 68.05 |
| 1.25 | 49.44 | 91.12 |

### Example 5—Cell Culture and Virulence Determination of Lipopolysaccharide from Different Bacteria

Reporter gene cell lines, human HEK 293T cells, were purchased from Invivogen of San Diego, Calif. The HEK 293T cells were stably transfected with at least two exogenous genes, a TLR4 structural gene, and a SEAP reporter gene, which is under the control of NFkB transcriptional factors. The cell line is named here as TLR4-SEAP. The reporter gene encodes a secreted enzyme, called embryonic alkaline phosphatase or SEAP. The SEAP reporter is placed under the control of the interferon-β minimal promoter fused to five NFkB and AP-1-binding sites. Furthermore, the TLR4-SEAP cell line also contains a CD14 co-receptor gene, which is needed to transfer LPS to TLR4 receptors. The recombinant TLR binds its ligand, or distinct pathogen-associated molecule, initiates a chain of responses, leading to recruitment of NFkB and AP1 transcription factors to the reporter gene promoter, which induce expression of SEAP.

Cell culture and treatment: 500,000 gene reporter cells were grown and maintained in 15 ml growth medium, comprised of DMEM medium supplemented with 10% fetal calf serum in T75 flasks for three days at 37° C., 5% CO₂, and 95% humidity. For treatment, wells of a 96-well plate were seeded with 10,000 cells/well in 100 μL of growth medium. The cells were incubated for 72 hours at 37° C., 5% CO₂, and 95% humidity until day 4. On day 4, medium was changed to assay medium (90 μl), which is the DMEM medium without fetal calf serum. LPS, bacteria and the culture medium of bacterial growth, as described in EXAMPLE 1, were first resolved or mixed with the assay medium. 10 μl of the bacteria, LPS and culture medium of bacterial growth were added to the TLR4-SEAP cells. Samples were taken 24 hours later, following addition of LPS, bacteria, and culture medium. Expression of the reporter gene (SEAP) was quantified with a commercially available kit (SEAP Reporter Gene Assay of Cayman Chemical Co., Ann Arbor, Mich.).

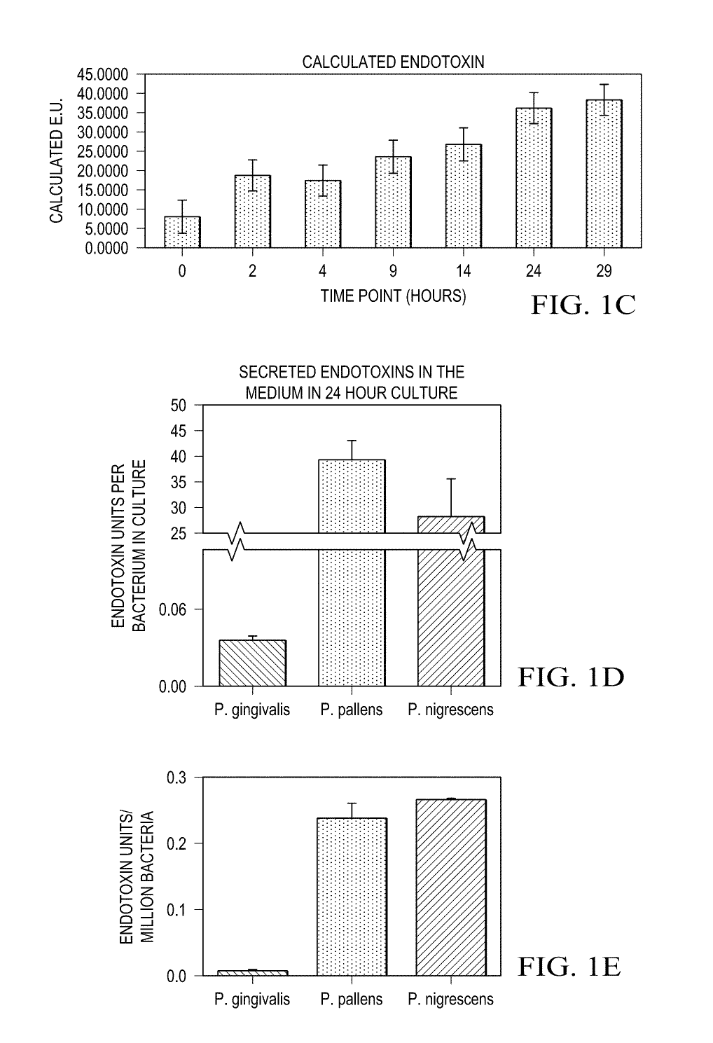

EC50 was calculated using GraphPad Prism software (GraphPad Software, La Jolla, Calif.). Samples with lower EC50 are more potent in activating the TLR4 reporter gene than those with higher EC50. As shown in FIG. 2A, LPS from E. coli has lower EC50 than P. gingivalis, thus, was far more potent than P. gingivalis (Pg). Salmonella Minnesota LPS is not as potent as that of E. coli, but is far more potent than those of P. gingivalis LPS 1690 and 1435. Each species of bacteria produces multiple forms of LPS. Each form of LPS from the same species of bacteria has different potency in stimulating TLR4-downstream signaling pathways. For example, Pg 1690 LPS is more potent than Pg1435/50. LPS is a component in bacterial cell walls. Likely, E. coli cell wall is more virulent in inducing production of proinflammatory cytokines in host cells than P. gingivalis when they are in direct contact with host blood cells. P. gingivalis had far higher EC50 than P. pallens and P. nigrescens as shown in FIG. 2B in stimulating TLR4 reporter gene expression, suggesting that P. pallens and P. nigrescens are more likely to promote production of proinflammatory cytokines in host cells than P. gingivalis.

Bacteria release LPS into the supernatant of bacterial culture. As shown in FIG. 2C, the supernatant of P. pallens has an EC50 that is similar to that of P. nigrescens, but far lower than that of P. gingivalis, in stimulating expression of TLR4 reporter gene. Again, those results imply that the products of P. pallens and P. nigrescens are more likely to promote production of proinflammatory cytokines in host cells than those of P. gingivalis.

### Example 6—Development of Technology for Inhibition of LPS Effects on Stimulating Expression of NFkB-Regulated Reporter Genes

Stannous fluoride is a leading anti-gingivitis technology in P&G toothpaste products. Tests were conducted to understand whether stannous fluoride could reduce LPS's ability to trigger proinflammatory responses in host cells. TLR4-SEAP reporter cells were prepared using the same conditions as described in EXAMPLE 5 in the presence or absence of LPS. Production of SEAP was quantified also as described in EXAMPLE 5.

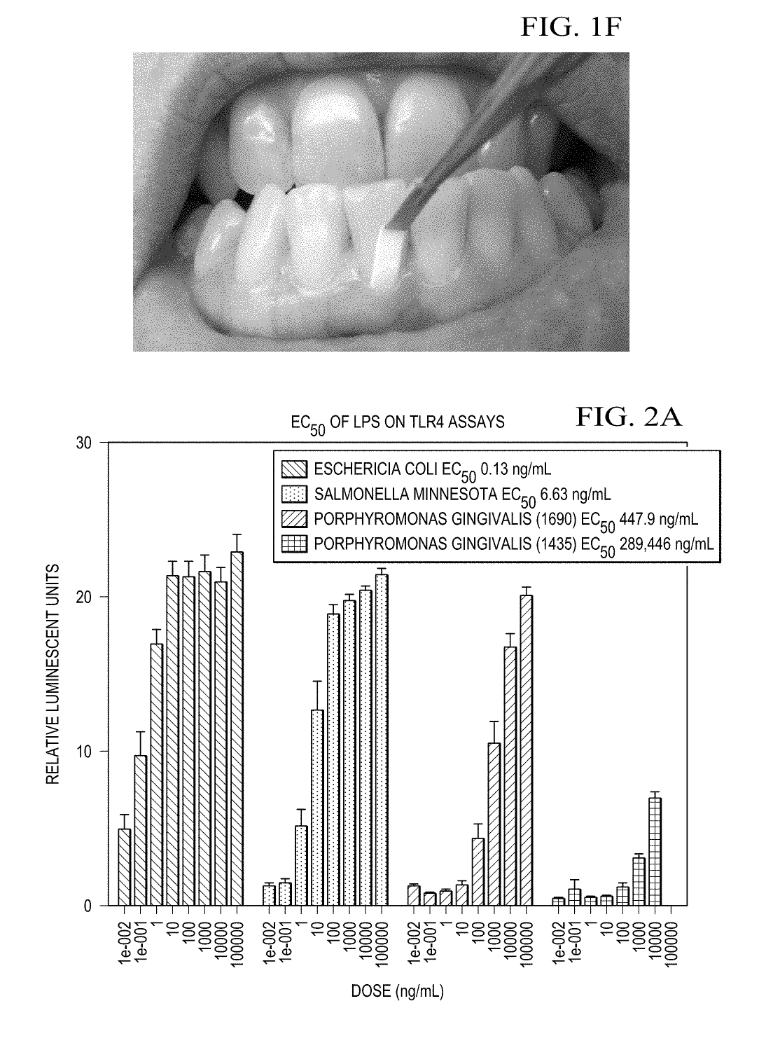

FIG. 3 shows the effect of stannous at various concentrations from 62.5 uM to 1,000 uM on 100 ng/ml E. coli LPS, as reported by activation of TLR-4. At stannous concentrations of 500 uM or higher, the level of E. coli induction of TLR-4 was decreased.

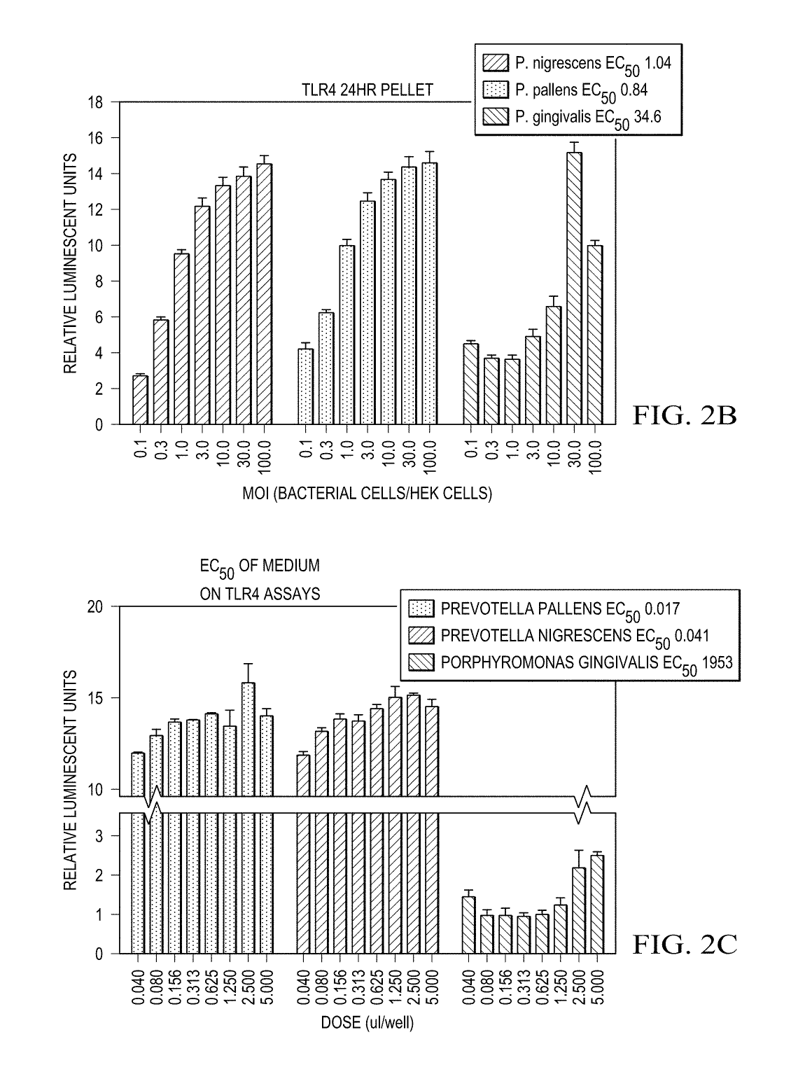

FIG. 4 shows the effects of stannous at various concentrations from 62.5 uM to 1,000 uM on P. gingivalis LPS, as reported by activation of TLR-2. At stannous concentrations of 1000 uM, the level of P. gingivalis induction of TLR-2 was decreased.

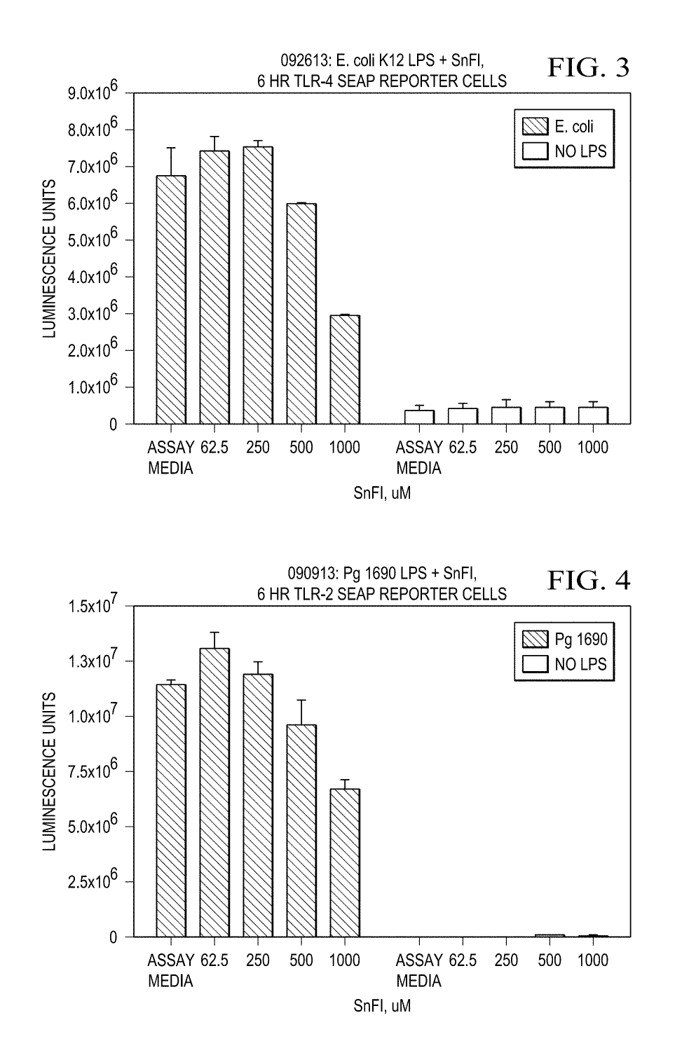

The data in FIG. 5 shows reduction of LPS activity by the stannous ion, from a stannous fluoride salt. The data showed that stannous fluoride, at 1.6 mM and 3.2 mM, reduce about 50% of P. gingivalis LPS (500 ng/ml) activation on the TLR4 reporter system (One asterisk means P<0.05, two asterisks mean P<0.01).

### Example 7—Cell Culture and Virulence Determination of EC50 in Activation of TLR4-SEAP Signal Transduction by Clinical Samples

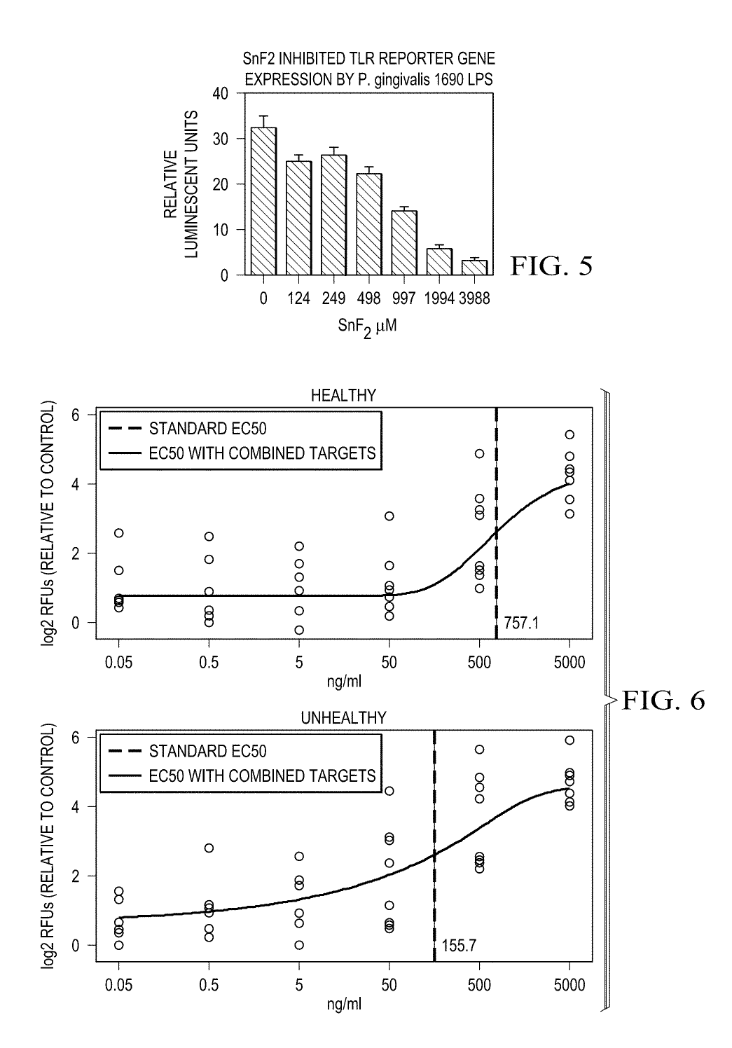

The method described in EXAMPLE 5 is effective at determining the potency of LPS from different bacteria. The same method was used to determine the EC50 of clinical samples, as described in EXAMPLE 2. As shown in FIG. 6, dental plaques from unhealthy sites had a smaller EC50 than those from healthy sites, suggesting the dental plaques from unhealthy sites contain more virulence factors.

The same method described in EXAMPLE 5 was used to examine the clinical samples in another study. A clinical study was conducted to evaluate sample collection methods and measurement procedures. It was a controlled, examiner-blind study. Forty panelists met the inclusion criteria, wherein in order to be included in the study, each panelist must:
Provide written informed consent to participate in the study;
Be 18 years of age or older;
Agree not to participate in any other oral/dental product studies during the course of this study;
Agree to delay any elective dentistry (including dental prophylaxis) until the study has been completed;
Agree to refrain from any form of non-specified oral hygiene during the treatment periods, including but not limited to the use of products such as floss or whitening products;
Agree to return for all scheduled visits and follow study procedures;
Must have at least 16 natural teeth;
Be in good general health, as determined by the Investigator/designee based on a review of the health history/update for participation in the study.

For Unhealthy Group (high bleeder group):
Have at least 20 bleeding sites (sites with a score of 1 or 2 on the GBI index); Have minimum 3 sampling sites with bleeding and pocket depth >3 mm but not deeper than 4 mm;
Have minimum 3 sampling sites without bleeding and with pocket depth <2 mm

For Healthy Group (low bleeder group):
Have maximum 3 bleeding sites (sites with a score of 1 or 2 on the GBI index);
No pockets deeper than 2 mm. Twenty (20) panelists were qualified as healthy—with up to 3 bleeding sites and with all pockets less than or equal to 2 mm deep and twenty (20) panelists were qualified as unhealthy—with greater than 20 bleeding sites with at least 3 pockets greater than or equal to 3 mm but not deeper than 4 mm with bleeding, and at least 3 pockets less than or equal to 2 mm deep with no bleeding for sampling. All panelists had up to 6 sites identified as “sampling sites.” The “sampling sites” had supragingival and subgingival plaque collected at Baseline, Week 2 and Week 4. Subgingival plaque samples were taken from a gingival sulcus from the pre-identified sites. Prior to sample collection, the site had supragingival plaque removed with a curette. The site was dried and subgingival plaque samples were collected with another dental curette (e.g., Gracey 13/14, 15/16, 11/12, 7/8, ½.) Each Gracey curette is designed to adapt to a specific area or tooth surface. For example, Gracey 13/14 is designed to adapt to the distal surfaces of posterior teeth. Samples from each site were placed in a pre-labeled 2.0 ml sterile tube containing 300 μl of DPBS buffer with about 50 of sterile 1 mm glass beads. Samples were stored at 4° C. The subgingival samples were stored at −80° C. until analyzed. The samples were thawed at room temperature and dispersed in a TissueLyser II (Qiagen, Valencia, Calif., USA) at 30 shakes per second for 3 min. Protein concentrations of the dispersed subgingival samples were measured using a Pierce microBCA Protein kit (ThermoFisher Scientific, Grand Island, N.Y., USA) following the manufacturer's instruction.

Oral lavage samples were collected at wake up (one per panelist) by rinsing with 4 ml of water for 30 seconds and then expectorating the contents of the mouth into a centrifuge tube. These samples were frozen at home until they were brought into the site in a cold pack. Each panelist collected up to 15 samples throughout the study. Saliva samples were frozen at −70° C. from submission.

All panelists were given investigational products: Crest® Pro-Health Clinical Gum Protection Toothpaste (0.454% stannous fluoride) and Oral-B® Indicator Soft Manual Toothbrush. Panelists continued their regular oral hygiene routine, and did not use any new products starting from the baseline to the end of four week treatment study. During the four week treatment period, panelists brushed their teeth twice daily, morning and evening, in their customary manner using the assigned dentifrice and soft manual toothbrush.

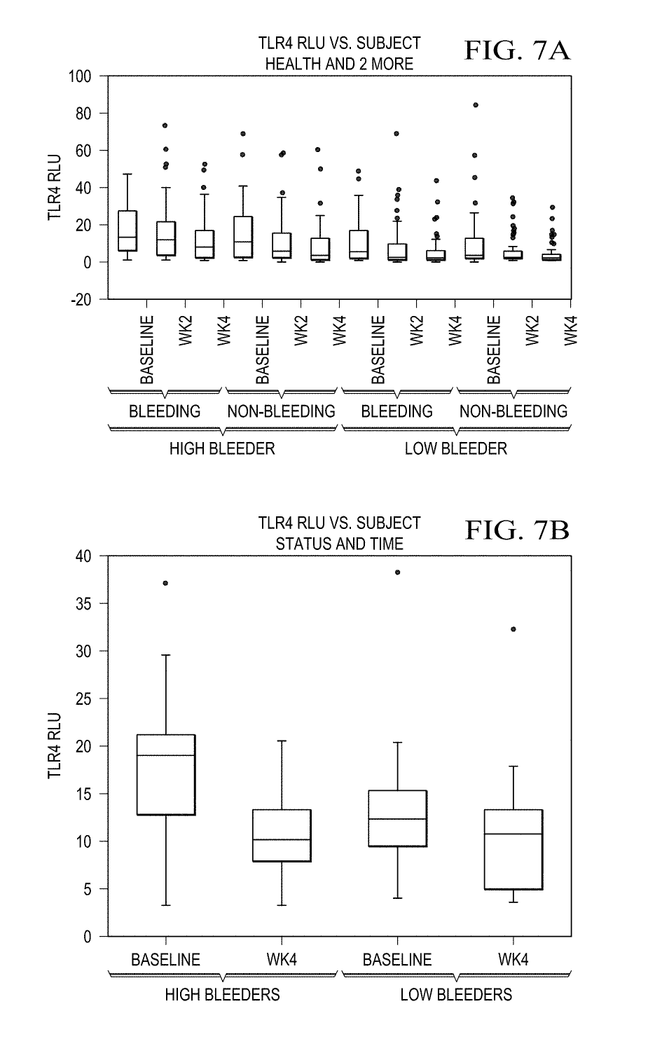

The subgingival plaques from the above clinical study were applied to the TLR4 reporter cells in a procedure as described in EXAMPLE 5. FIG. 7A shows the results of a four-week study of 40 panelists going from baseline out over four weeks of treatment with Crest ProHealth Clinical toothpaste. The subgingival plaque samples in bleeding sites on the high bleeders group stimulated high expression of TLR4 reporter gene. More virulence in a sample elicits higher RLU (relative luminescent units) readings in the TLR4 reporter gene assay. As shown in FIG. 7A, the baseline samples of the high bleeders group had higher RLU than those of the low bleeders on both the bleeding and non-bleeding sites. After treatment with Crest® Pro-Health Clinical Gum Protection Toothpaste (0.454% stannous fluoride) and Oral-B® Indicator Soft Manual Toothbrush for four weeks, the virulence was reduced at week 4 in both high and lower bleeders groups at both bleeding and non-bleeding sites.

The oral lavage samples were applied to the TLR4 reporter cells in a procedure as described in EXAMPLE 5. As shown in FIG. 7B, oral lavage (Healthy vs. Gingivitis) samples were evaluated in the TLR4-SEAP reporter assay. The baseline samples of the high bleeders group had higher RLU than those of the low bleeders. After treatment with Crest® Pro-Health Clinical Gum Protection Toothpaste (0.454% stannous fluoride) and Oral-B® Indicator Soft Manual Toothbrush for four weeks, the virulence was reduced at week 4 in the high bleeder group.

### Example 8—Cell Culture and Virulence Determination of Lipoteichoic Acids from Different Bacteria

The reporter gene cell lines, human HEK 293T cells, were purchased from Invivogen of San Diego, Calif. The HEK 293T cells were stably transfected with at least two exogenous genes, a TLR2 structural gene, and SEAP reporter gene which is under the control of NFkB transcriptional factors. The cell line is named here as TLR2-SEAP. The reporter gene encodes a secreted enzyme, called embryonic alkaline phosphatase or SEAP. The SEAP reporter is placed under the control of the interferon-β minimal promoter fused to five NFkB and AP-1-binding sites. Furthermore, a CD14 co-receptor gene was transfected into the reporter gene cells expressing TLR2, as CD14 has been identified as a co-receptor for TLR2 ligands to enhance the TLR response. The CD14 co-receptor is needed to transfer LTA to TLR2 receptors. The recombinant TLR2 binds its ligand, or distinct pathogen-associated molecule, initiates a chain of responses, leading to recruitment of NFkB and AP1 transcription factors to the reporter gene promoter, which induce expression of SEAP.

Cell culture and treatment: 500,000 gene reporter cells were grown and maintained in 15 ml growth medium, comprising DMEM medium supplemented with 10% fetal calf serum in T75 flasks for three days at 37° C., 5% CO₂, and 95% humidity. For treatment with LTA, wells of a 96-well plate were seeded with 10,000 cells/well in 100 μL of growth medium. The cells were incubated for 72 hours at 37° C., 5% CO₂, and 95% humidity until day 4. On day 4, medium (100 μL) was changed to DMEM medium without fetal calf serum. LTA, LPS and bacterial cells, as described in EXAMPLE 7, were added. Samples were taken 24 hours later, following addition of samples. Expression of the reporter gene (SEAP) was quantified with a commercially available kit (SEAP Reporter Gene Assay of Cayman Chemical Co., Ann Arbor, Mich.).

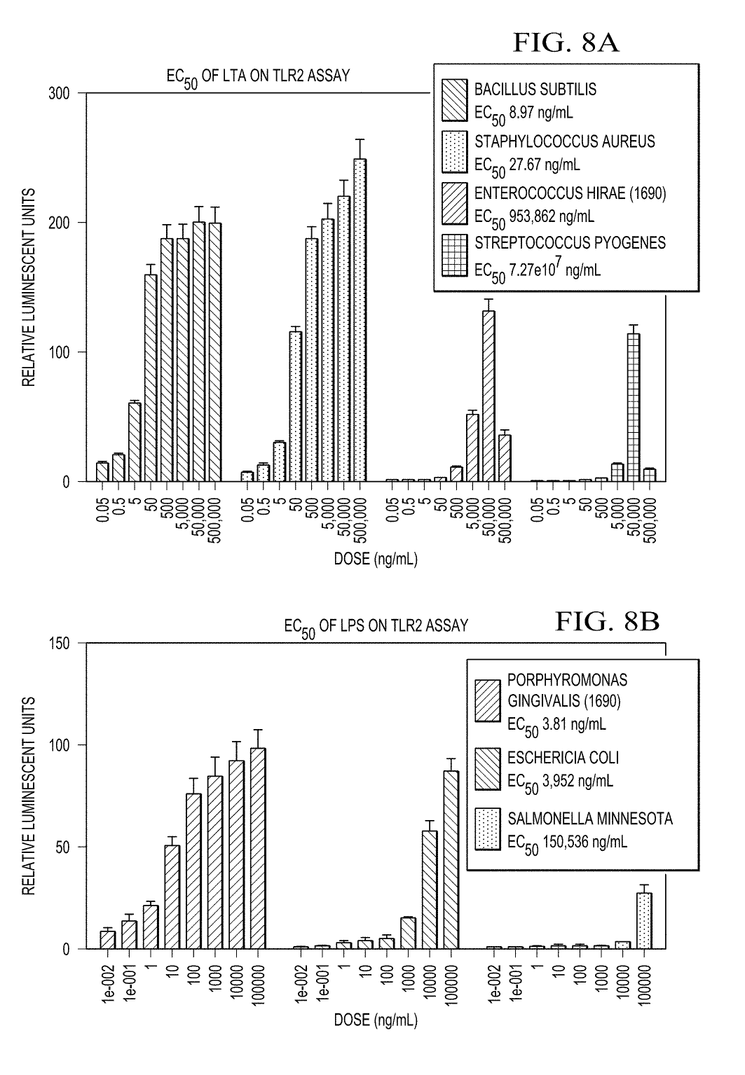

As shown in FIGS. 8A, 8B, 8C and 8D, LTA, LPS, bacteria and the supernatant of bacterial culture could bind to TLR2 and activate TLR2 downstream signaling pathways in a dose-dependent manner. As shown in FIG. 8A, B. subtilis (BS) LTA is more potent than that of Enterococccus hirae. As shown in FIG. 8B, P. gingivalis LPS also activated expression of the TLR2 reporter gene. For example, Pg1690, as shown in FIG. 8B, activated TLR2-SEAP signal transduction, and stimulated SEAP production. But as shown in FIG. 8B, E. coli LPS did not activate the TLR2-SEAP reporter cells. It should also be noted that P. pallens, P. nigrescens and P. gingivalis have similar EC50 in stimulating expression of TLR2 reporter gene (FIG. 8C). However, the released TLR2 ligands from the three different bacteria have very different EC50 on activation of TLR2 reporter gene (FIG. 8D).

### Example 9—Cell Culture and Virulence Determination of EC50 in Activation of TLR2-SEAP Signal Transduction by Clinical Samples

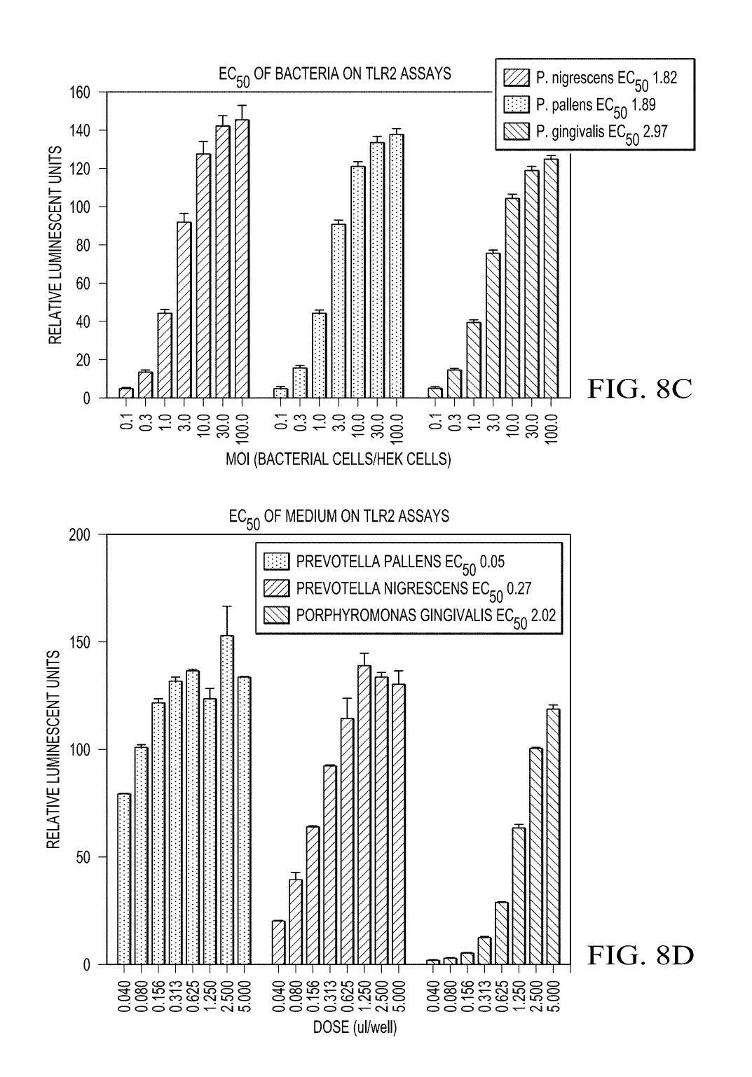

The method described in EXAMPLE 8 is effective in determining the EC50 of LTA and other TLR2 ligands from different bacteria. The same method was used to determine the EC50 of clinical samples, as described in EXAMPLE 2. As shown in FIG. 9, dental plaques from unhealthy (bleeding) sites had smaller EC50 than those from healthy (non-bleeding) sites, suggesting the dental plaques from unhealthy sites contain more virulence factors.

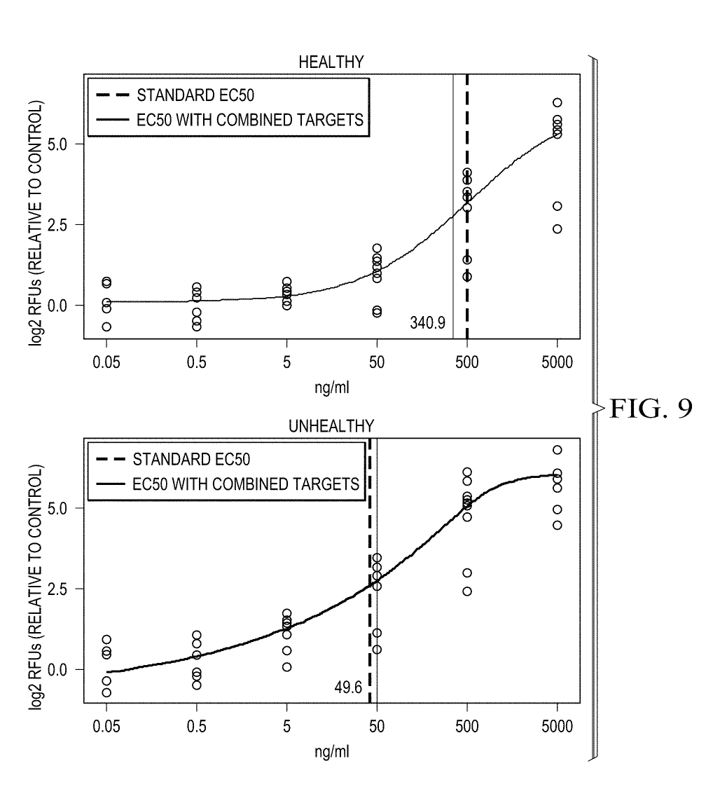

Clinical samples as described for FIG. 7A of EXAMPLE 7 were examined using the TLR2-SEAP reporter gene assay. The results are shown in FIG. 10A. The subgingival samples in unhealthy (bleeding) sites from the unhealthy group (high bleeders) had more virulence factors than other sites. The baseline samples of the high bleeders group had higher RLU than those of the low bleeders on both the bleeding and non-bleeding sites. After treatment with Crest® Pro-Health Clinical Gum Protection Toothpaste (0.454% stannous fluoride) and Oral-B® Indicator Soft Manual Toothbrush for four weeks, the virulence was reduced at week 4 in both high and low bleeders groups at both bleeding sites.

The clinical samples as described for FIG. 7B of EXAMPLE 7 were examined using the TLR2-SEAP reporter gene assay. As shown in FIG. 10B, oral lavage (Healthy vs. Gingivitis) was evaluated. After treatment with Crest® Pro-Health Clinical Gum Protection Toothpaste (0.454% stannous fluoride) and Oral-B® Indicator Soft Manual Toothbrush for four weeks, the virulence was reduced at week 4 in the high bleeder group.

### Example 10—Cell Culture and Virulence Determination of Flagellins from Different Bacteria

The reporter gene cell lines, human HEK 293T cells, were purchased from Invivogen of San Diego Calif. The HEK 293T cells were stably transfected with two exogenous genes, a TLR5 structural gene, and SEAP reporter gene which is under the control of NFkB transcriptional factors. The cell line was named as TLR5-NFkB-SEAP. The reporter gene encodes a secreted enzyme, called embryonic alkaline phosphatase or SEAP. The SEAP reporter is placed under the control of the interferon-β minimal promoter fused to five NFkB and AP-1-binding sites. The recombinant TLR5 binds to its ligand, or distinct pathogen-associated molecule, and initiates a chain of responses leading to recruitment of NFkB and AP1 transcription factors to the reporter gene promoter, which induce expression of SEAP.

Cell culture and treatment: 500,000 gene reporter cells were grown and maintained in DMEM medium supplemented with 10% fetal calf serum in T75 flasks for three days at 37° C., 5% CO₂, and 95% humidity. For treatment with flagellin, wells of a 96-well plate were seeded with 10,000 cells/well in 100 μL of growth medium. The cells were incubated for 72 hours at 37° C., 5% CO₂, 95% humidity until day 4 after cells were seeded onto wells of a 96-well plate. On day 4, medium (100 μL) was changed to DMEM medium without fetal calf serum. S. subtilis and S. aureus flagellin were added at a range of concentration from 0.97 ng to 1 μg/ml. Samples were taken at 6 and 24 hours later after adding flagellin. Expression of reporter gene (SEAP) was quantified with a commercially available kit (SEAP Reporter Gene Assay of Cayman Chemical Co., Ann Arbor, Mich.).

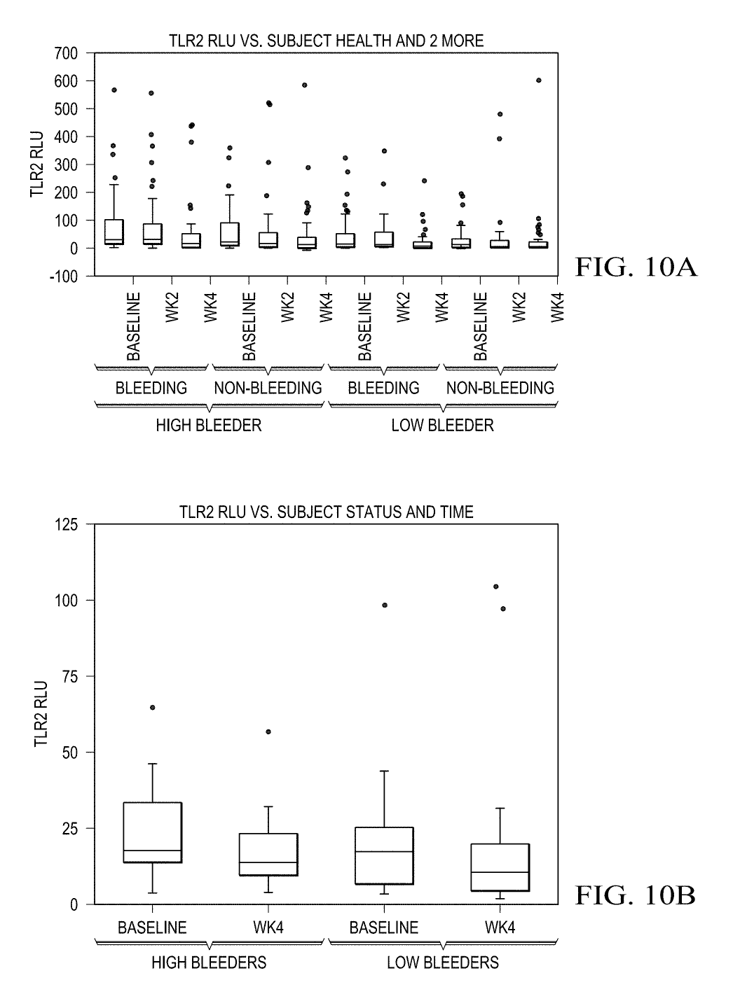

FIG. 11 shows both flagellins (B. subtilis and P. aeruginosa) could activate TLR5 downstream signaling pathways. Flagellin from B. subtilis stimulated TLR5-SEAP signaling transduction in a dose-dependent manner. The EC50 of flagellin on TLR5 was 0.02 ng/ml at 5 hours and 0.014 ng/ml at 23 hours. The EC50 of flagellin from Pseudomonas aeruginosa on TLR5 was 0.0049 at 5 hours and 0.0019 at 23 hours. Flagellin from P. aeruginosa was more potent on stimulating TLR5 signaling pathways.

### Example 11—Cell Culture and Virulence Determination of LPS from Different Bacteria in THP1 Dual Reporter Cells

Bacterial cell wall and membrane components are recognized by TLR2. TLR2 recognizes the microbial motifs PGN (peptidoglycan)/lipoproteins/dectin and LPS. TLR1 and TLR6 form heterodimers with TLR2 and bind to triacylated lipoproteins and diacylated lipoproteins, respectively. THP1 NFkB-SEAP and IRF-Lucia™ Reporter Monocytes were purchased from Invivogen, San Diego, Calif. THP1-Dual cells were derived from the human THP-1 monocyte cell line by stable integration of two inducible reporter constructs. THP1-Dual cells feature the Lucia gene under the control of an ISG54 (interferon-stimulated gene) minimal promoter in conjunction with five interferon-stimulated response elements. THP1-Dual cells also express a SEAP reporter gene driven by an IFN-b minimal promoter fused to five copies of the NF-kB consensus transcriptional response element and three copies of the c-Rel binding site. As a result, THP1-Dual cells allow the simultaneous study of the NFkB pathway, by monitoring the activity of SEAP, and the interferon regulatory factor (IRF) pathway, by assessing the activity of Lucia (IRF-Luc). Both reporter proteins are readily measurable in the cell culture supernatant. This THP-1 cell line possesses functional TLR1, TLR2, TLR4, TLR5, TLR6 and TLR8, purchased from Invivogen. TLR4 senses LPS from Gram-negative bacteria while TLR5 recognizes bacterial flagellin from both Gram-positive and Gram-negative bacteria, TLR8 detects long single-stranded RNA.

Culture and treatment: The THP1-dual cells were cultured in 15 ml growth medium (RPMI 1640 with 10% heat-inactivated fetal bovine serum) in a T75 flask at 37° C. and 5% CO₂. Cells were passed every 3 to 4 days by inoculating 300,000-500,000 cells/ml into a fresh T75 flask with 15 ml of fresh growth medium. To determine the effect of bacterial components on reporter gene expression, wells in 96-well plates were seeded at 100,000 cells in 90 μl of growth medium. 10 μl of bacterial wall and membrane components, or heat-killed whole bacteria, were added to each well. After incubation for 18 hours at 37° C. and 5% CO₂, secreted luciferase and SEAP were quantified with commercially available assay kits (QUANTI-Luc of Invivogen, San Diego, Calif. for luciferase; SEAP Reporter Gene Assay of Cayman Chemical Co., Ann Arbor, Mich. for SEAP).

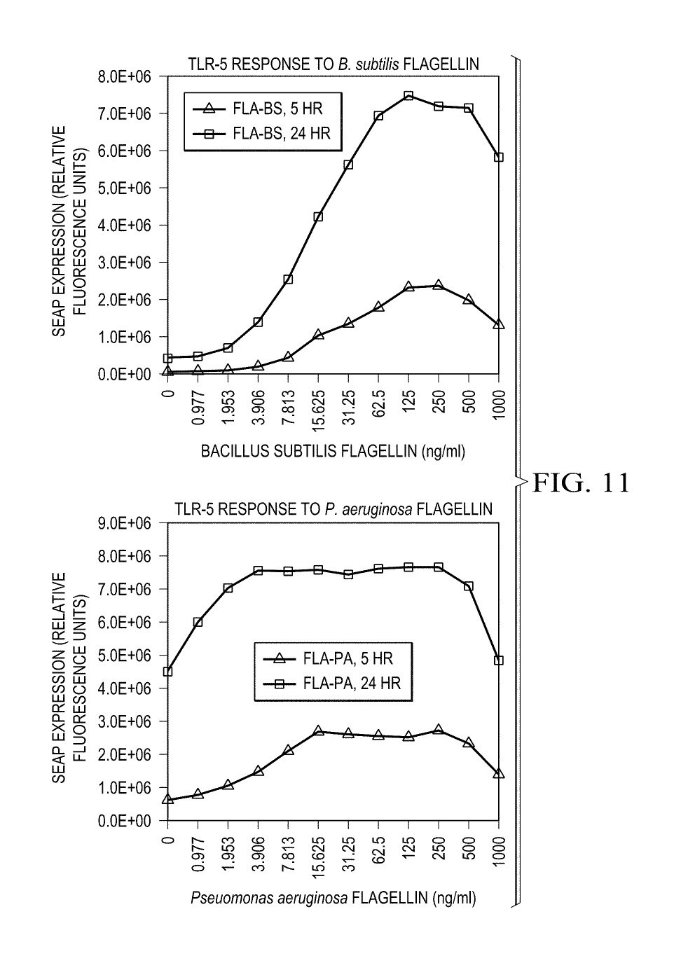

DHP1-dual reporter cells were treated with three different preparations of LPS as shown in FIG. 12A. All three LPS (ng/ml) activated production of NFkB-SEAP reporter genes in a dose-dependent manner. In addition, Pg 1690 LPS and E. coli LPS also stimulated expression of the IRF-luciferase reporter gene. TLR4 ligands, upon binding to TLR4 receptors, activate at least two signaling pathways. One is a common pathway NFkB-SEAP, which can be activated by all TLR ligands upon binding to their specific receptors. For example, TLR2 ligand, LTA, can bind to TLR2 receptors and activate the NFkB-SEAP signaling pathway. Similarly, TLR4 ligand, LPS, upon binding to TLR4 receptors, also is able to activate the NFkB-SEAP signaling transduction. As shown in FIG. 12A, E. coli LPS is a more potent ligand than P. gingivalis 1690 LPS on activation of both NFkB-SEAP and IRF-luciferase signaling transduction. THP-1 cells produce several functional TLR receptors. And all TLR receptors can activate the NFkB pathway, thus promoting expression of the NFkB-SEAP reporter gene. The reading of NFkB-SEAP is the collective actions of all TLR receptors, such as TLR2, TLR1, TLR6 and TLR4. All LPS from different bacteria stimulated NFkB-SEAP reporter gene. IRF-luciferase reporter gene, on the other hand, is driven by a limited number of TLR receptors, primarily TLR3, TLR4, TLR7, TLR8 and TLR9. Both P. gingivalis LPS 1690 and E. coli LPS stimulated expression of IRF-luciferase in a dose-dependent fashion.

The THP-1 reporter cells were used to examine the clinical samples as described for FIG. 7B of EXAMPLE 7. As shown in FIG. 12B, oral lavage (Healthy vs. Gingivitis) was evaluated using the IRF-Luc reporter gene in THP-1 cells. After treatment with Crest® Pro-Health Clinical Gum Protection Toothpaste (0.454% stannous fluoride) and Oral-B® Indicator Soft Manual Toothbrush for four weeks, the virulence was reduced at week 4 in both high and lower bleeders groups.

The THP-1 reporter cells were used to examine the clinical samples as described for FIG. 7A of EXAMPLE 7. As shown in FIG. 12C, the subgingival Plaque (Healthy vs. Gingivitis) was examined using the NFkB reporter gene in THP-1 cells. The baseline samples of the high bleeders group had higher RLU than those of the low bleeders. After treatment with Crest® Pro-Health Clinical Gum Protection Toothpaste (0.454% stannous fluoride) and Oral-B® Indicator Soft Manual Toothbrush for four weeks, the virulence was reduced at week 4 in the bleeding sites in both high and lower bleeders groups.

### Example 12—Cell Culture and Virulence Determination of LTA from Different Bacteria and Biofilms in THP1 Dual Reporter Cells

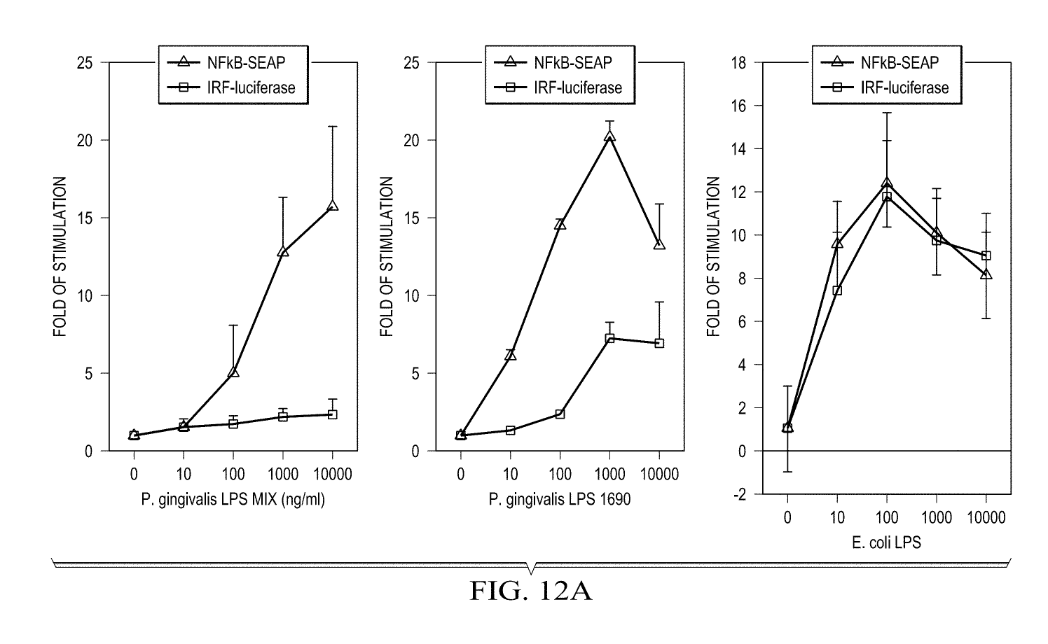

THP1 dual reporter cells also express TLR2, TLR1 and TLR6 receptors. Bacterial cell wall and some membrane components are recognized by TLR2, TLR1 and TLR6. TLR2 recognizes the microbial motifs PGN (peptidoglycan)/lipoproteins/dectin and LPS. To determine whether LTA from different bacteria have different effects on stimulating NFkB-SEAP reporter gene expression in the THP1 dual reporter cells, the cells were prepared and treated in the same procedures as described in EXAMPLE 11. As shown in FIG. 13, LTA from both B. subtilis and S. aureus had similar potency in promoting reporter gene expression in the THP1 dual reporter cells.

### Example 13—Distinguishing Bacterial Components from Different Bacteria in Human Primary Gingival Epithelial Cells

Primary human gingival epithelial cells were purchased from Zen-bio (Research Triangle Park, N.C.), and maintained in 15 ml of growth medium (CellnTec medium supplemented with CellnTec Growth Supplement, purchased from CellnTec Advanced Cell Systems AG, Bern, Switzerland) in T75 flasks at 37° C. under a 5% CO2 atmosphere. As the experiment was done at 24h, 48h and 72h time point and with assay media, CellnTec medium alone or with supplements, six 96 well plates were seeded with 7,500 cells/well in 100 μl of CELLnTEC growth medium at 37° C. under a 5% CO2 atmosphere. The growth medium was changed to assay medium right before adding LPS or bacterial DNA. For example, if the assay medium was CellnTec medium without supplements, 100 μl of CellnTec medium was added without supplements in each well. If the assay medium was the growth medium, 100 μl of growth medium was added to each well. The P. gingivalis LPS and bacterial DNA were added to the cells. At 24 h, 48 h and 72 h, medium was collected for analysis. Cytokines were measured using Elisa kits from Meso Scale Discovery (Rockville, Md.), as per the manufacturer's instructions.

As shown in TABLE 4, human primary gingival epithelial cells were treated with P. gingivalis DNA at 0, 0.3, 1 and 2 μg/ml. The cultures were harvested at 24 and 48 hours after treatment and six proinflammatory cytokines (interferon-γ, IL-1β, IL-2, IL-10, IL-12p70 and TNF-α) were analyzed using ELISA kits from Meso Scale Discovery. As shown in TABLE 4, expression of interferon-γ, IL-1β, IL-2, IL-10, IL-12p70 and TNF-α was low, or almost undetectable (each value is the mean of three replicate in one experiment). They were not viable biomarkers in distinguishing bacterial DNA. The results in TABLE 4 illustrate the lack of inflammatory response from human gingival epithelial cells, thus demonstrating the need to use an engineered cell.

TABLE 4
Screening of Human Gingival Epithelial Cells for
Inflammatory Markers treated with P. gingivalis DNA
Production of cytokines (pg/ml) in primary human
gingival epithelial cells treated with P. gingivalis
genomic DNA

| | P. | | | | P. | | |
|---|---|---|---|---|---|---|---|
| | gingivalis | | | | gingivalis | | |
| | genomic | 24 | 48 | | genomic | 24 | 48 |
| | DNA | hours | hours | | DNA | hours | hours |
| Interferon | 0 ug | 0 | 0.25 | IL-1 | 0 ug | 0 | 0.5 |
| gamma | 0.3 ug | 0 | 0.33 | beta | 0.3 ug | 0 | 0.33 |
| | 1 ug | 0 | 0 | | 1 ug | 0 | 0 |
| | 2 ug | 0.33 | 0.67 | | 2 ug | 0 | 0 |
| IL-10 | PG DNA | 0.5 | 1 | IL- | PG DNA | 0 | 0.25 |
| | 0 ug | | | 12p70 | 0 ug | | |
| | PG DNA | 0.67 | 0.33 | | PG DNA | 0.67 | 0 |
| | 0.3 ug | | | | 0.3 ug | | |
| | PG DNA | 0 | 0 | | PG DNA | 0 | 0.67 |
| | 1 ug | | | | 1 ug | | |
| | PG DNA | 0.33 | 0.33 | | PG DNA | 0 | 0.33 |
| | 2 ug | | | | 2 ug | | |
| IL-2 | PG DNA | 0.5 | 1 | TNF- | PG DNA | 1 | 1.5 |
| | 0 ug | | | alpha | 0 ug | | |
| | PG DNA | 0 | 0.67 | | PG DNA | 1.67 | 1.33 |
| | 0.3 ug | | | | 0.3 ug | | |
| | PG DNA | 0.33 | 1 | | PG DNA | 0.67 | 1 |
| | 1 ug | | | | 1 ug | | |
| | PG DNA | 0.33 | 0.67 | | PG DNA | 1.33 | 1.33 |
| | 2 ug | | | | 2 ug | | |

Human primary gingival epithelial cells were treated with P. gingivalis LPS at 0, 0.3, 1 and 2 μg/ml in the procedures described above. As shown in TABLE 5, again, expression of interferon-γ, IL-1β, IL-2, IL-10, IL-12p70 and TNF-α was low, or almost undetectable. They were not viable biomarkers in distinguishing bacterial LPS in primary human gingival epithelial cells. The results in TABLE 5 further illustrate how human gingival cells were not sufficiently sensitive to bacterial virulence factors for an assay, and thus the need to utilize a cell line capable of demonstrating a dose dependent response to virulence factors.

TABLE 5
Screening of Human Gingival Epithelial Cells for Inflammatory
Markers treated with P. gingivalis LPS
Production of cytokines (pg/ml) in primary human gingival
epithelial cells treated with P. gingivalis LPS

| | | 24 h | 48 h | | | 24 h | 48 h |
|---|---|---|---|---|---|---|---|
| Interferon | PG lPS 0 ug | 0.00 | 0.25 | IL-1 | PG lPS 0 ug | 0.00 | 0.50 |
| gamma | PG LPS 0.3 ug | 0.00 | 0.00 | beta | PG LPS 0.3 ug | 0.00 | 0.67 |
| | PG LPS 1 ug | 0.33 | 0.33 | | PG LPS 1 ug | 0.00 | 0.00 |
| | PG LPS 2 ug | 0.00 | 0.00 | | PG LPS 2 ug | 0.00 | 1.00 |
| IL-10 | PG lPS 0 ug | 0.50 | 1.00 | IL- | PG lPS 0 ug | 0.00 | 0.25 |
| | PG LPS 0.3 ug | 0.67 | 0.67 | 12p70 | PG LPS 0.3 ug | 0.00 | 0.00 |
| | PG LPS 1 ug | 0.00 | 0.67 | | PG LPS 1 ug | 0.00 | 0.33 |
| | PG LPS 2 ug | 0.33 | 0.67 | | PG LPS 2 ug | 0.67 | 0.00 |
| IL-2 | PG lPS 0 ug | 0.50 | 1.00 | TNF- | PG lPS 0 ug | 1.00 | 1.50 |
| | PG LPS 0.3 ug | 0.33 | 0.67 | alpha | PG LPS 0.3 ug | 1.33 | 0.67 |
| | PG LPS 1 ug | 0.33 | 1.00 | | PG LPS 1 ug | 2.00 | 1.33 |
| | PG LPS 2 ug | 1.33 | 1.00 | | PG LPS 2 ug | 0.67 | 1.33 |

### Example 14—Gingivitis-Enriched Bacteria Reduced in Abundance in Six Weeks of Gingivitis Treatment

A randomized, two-group clinical study was conducted with 69 panelists (35 in the negative control group and 34 in the test regimen group). Panelists were 39 years old on average, ranging from 20 to 69, and 46% of the panelists were female. Treatment groups were well balanced, since there were no statistically significant (p≥0.395) differences for demographic characteristics (age, ethnicity, gender) or starting measurements for Gingival Bleeding Index (GBI); mean=29.957 with at least 20 bleeding sites, and Modified Gingival Index (MGI); mean=2.086. All sixty-nine panelists attended each visit and completed the treatment process. The following treatment groups were compared over a 6-week period:

Test regimen: Crest® Pro-Health Clinical Plaque Control (0.454% stannous fluoride) dentifrice, Oral-B® Professional Care 1000 with Precision Clean brush head and Crest® Pro-Health Refreshing Clean Mint (0.07% CPC) mouth rinse. Control regimen: Crest® Cavity Protection (0.243% sodium fluoride) dentifrice and Oral-B® Indicator Soft Manual toothbrush.

Dental plaques were collected from the same panelists in the test regimen in the clinical study as described in EXAMPLE 2. A supragingival sample was taken from each panelist with a sterile curette at the tooth/gum interface, using care to avoid contact with the oral soft tissue. Plaques were sampled from all available natural teeth (upper arch only) until no plaque was visible. Following sampling, the plaque samples were placed into a pre-labeled (panelist ID, sample initials, visit, and date) Eppendorf tube with 1 ml of PBS/Glycerol buffer and about 50 of sterile 1 mm glass beads, stored on ice until all samples were collected. The samples were then transferred to a −70° C. freezer for storage until further processing. Genomic DNA was isolated from supragingival plaque samples using QIAamp® genomic DNA kits (Qiagen, Germany) following manufacturer's instruction. Metasequencing was carried out at BGI Americas Corporation (Cambridge, Mass.). All data were analyzed at Global Biotech of Procter & Gamble Company in Mason, Ohio.

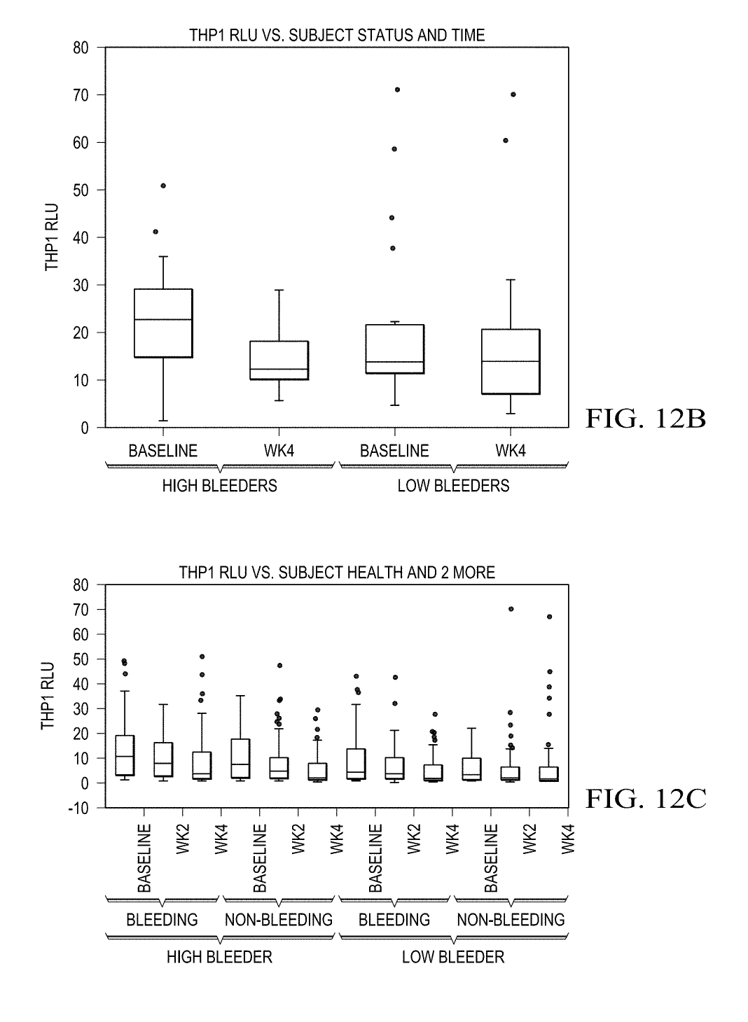

Clinical measurements: Bleeding sites (GBI) were decreased in the test regimen significantly on week 1, 3 and 6 in comparison to the control regimen (FIG. 14). Similarly, Inflammation (MGI) grades also decreased in the test regimen (FIG. 14).

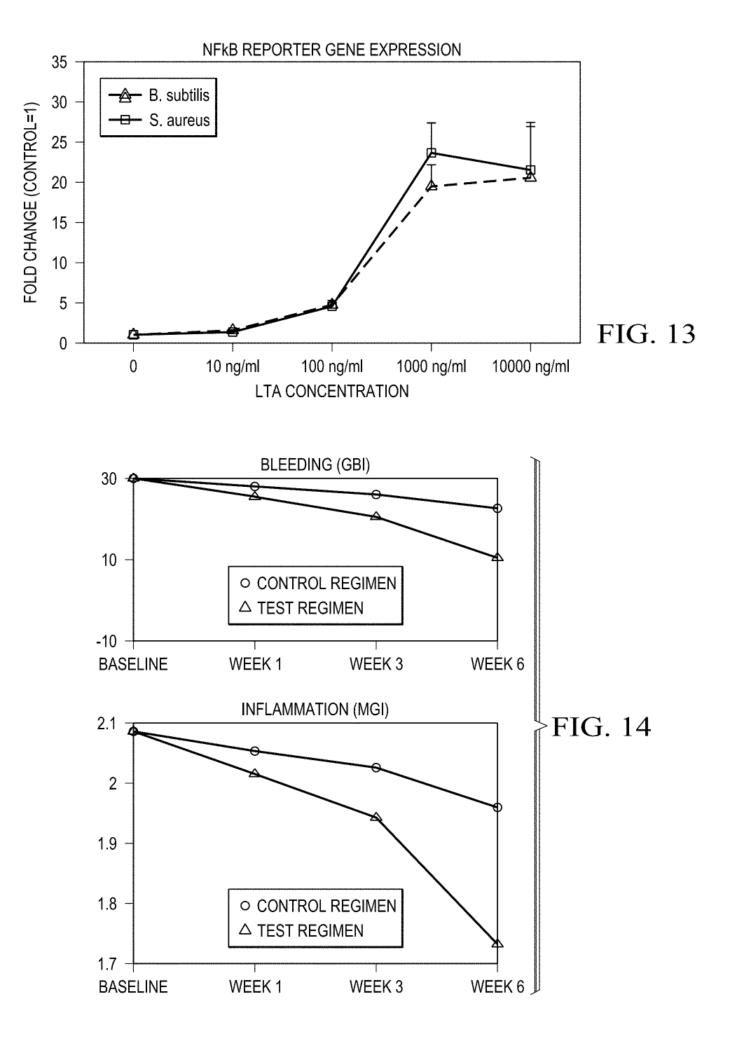

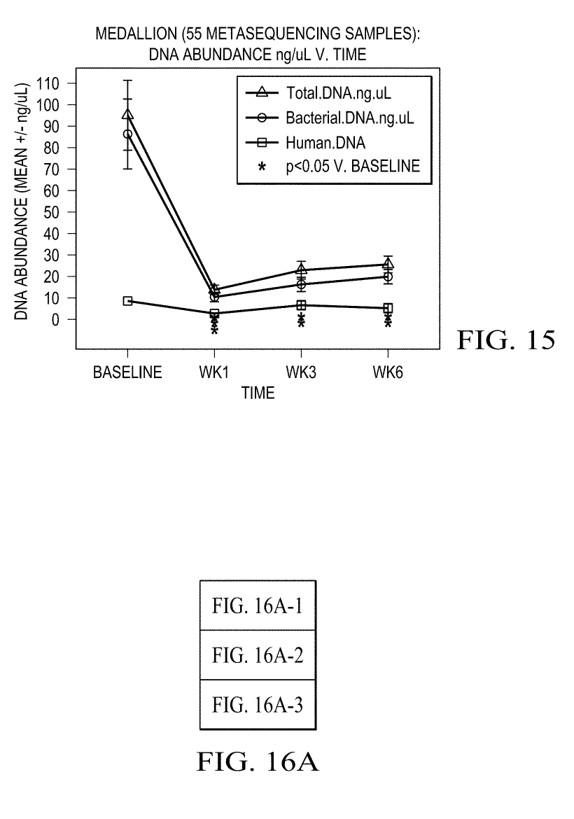

Genomic DNA of the supragingival plaques in the test regimen was sequenced. As shown in FIG. 15, abundance of certain bacteria in the supragingival plaques changed in the six week treatments. Certain bacteria, such as Porphyromonas sp oral taxon 279 and Prevotella pallens, were decreased in weeks 1 and 3 (FIG. 15). The amount of each bacterial species was plotted over the four time periods of the treatment. The amount of certain bacteria, such as Peptostreptococcus stomatis and Prevotella intermedia, was reduced during the six week of treatment as shown in FIG. 15. This is also shown in FIG. 16A-1, FIG. 16A-2, and FIG. 16A-3.

### Example 15—Production of Cytokines, Chemokines and Other Bioactive Proteins Decreased as Gingivitis Symptoms were Alleviated During 6 Week of Treatment

In the same clinical study as described in EXAMPLE 14, gingival-brush samples were collected from the same panelists as in EXAMPLE 14. Before sampling, panelists rinsed their mouths for 30 seconds with water. A dental hygienist then sampled the area just above the gumline using a buccal swab brush (Epicentre Biotechnologies cat.# MB100SP). The swab was immediately placed into 1 ml extraction buffer [PBS, 0.25M NaCl, 1×Halt™ Protease Inhibitor Single-Use Cocktail (Lifetechnologies, Grand Island, N.Y.)] in a 1.5 ml Eppendorf tube vortexed for 30 seconds, and immediately frozen on dry ice and stored in a −80 C freezer until analysis. The samples were taken out of freezer, thawed and extracted by placing the samples on a tube shaker for 30 minutes at 4° C. The tubes were centrifuged at 15000 RPM for 10 min in Eppendorf Centrifuge 5417R (Eppendorf, Ontario, Canada) to pellet any debris. The extract (800 μl) was analyzed for protein concentrations using the Bio-Rad protein assay (BioRad, Hercules, Calif.).

Forty proteins were measured in the gingival samples using V-PLEX Human Biomarker 40-Plex Kit (Meso Scale Diagnostics Rockville, Md.). The assay was performed following the manufacturer's instruction.

Among the proteins measured in the gingival samples, most proteins in the Proinflammatory Panel 1 (human), Cytokine Panel 1 (human), Chemokine Panel 1 (human), Angiogenesis Panel 1 (human), and Vascular Injury Panel 2 (human) had significant changes in their abundance during the 6-week treatment (TABLE 6). Those include FN-γ, IL-1β, IL-2, IL-4, IL-6, IL-8, IL-10, IL-12p70, IL-13, TNF-α, GM-CSF, IL-5, IL-16, IL-7, IL-12/IL-23p40, IL-1α, VEGF-A, IL-17A, IL-15, TNF-β, IL-8 (HA), MCP-1, MCP-4, Eotaxin, IP-10, MDC, Eotaxin-3, TARC, MIP-1α, MIP-1β, VEGF-C, VEGF-D, Tie-2, Flt-1/VEGFR1, PlGF, FGF (basic), SAA, CRP, VCAM-1, and ICAM-1.

TABLE 6
Changes in abundance of proteins in the gingival-brush samples

| | Mean | | | | α = 0.05 | | | |
|---|---|---|---|---|---|---|---|---|
| | Baseline | Week 1 | Week 3 | Week 6 | Baseline | Week 1 | Week 3 | Week 6 |
| ICAM-1 | 16.035 | 12.209 | 10.090 | 9.767 | A | B | B, C | C |
| IL-1α | 3.554 | 2.331 | 2.181 | 1.891 | A | A, B | B, C | C |
| IL-1β | 53.666 | 35.575 | 24.295 | 24.440 | A | B | C | C |
| TNF-β | 0.0013 | 0.0010 | 0.0008 | 0.0007 | A | B | C | C |
| IL-12p70 | 0.172 | 0.148 | 0.118 | 0.127 | A | A, B | C | B, C |
| IL-13 | 0.805 | 0.762 | 0.624 | 0.648 | A | A, B | C | B, C |
| IL-4 | 0.127 | 0.115 | 0.090 | 0.096 | A | A, B | C | B, C |
| IL-5 | 0.004 | 0.003 | 0.002 | 0.003 | A | B | C | B, C |
| CRP | 15.637 | 12.743 | 12.385 | 5.809 | A | A | A | B |
| Eotaxin | 0.077 | 0.064 | 0.059 | 0.059 | A | A, B | B | B |
| GM-CSF | 0.010 | 0.008 | 0.008 | 0.008 | A | B | B | B |
| IFN [symbol] | 0.530 | 0.446 | 0.378 | 0.386 | A | A, B | B | B |
| IL-10 | 0.875 | 0.490 | 0.423 | 0.244 | A | A, B | B | B |
| IL-15 | 0.005 | 0.003 | 0.003 | 0.003 | A | B | B | B |
| IL-16 | 0.466 | 0.345 | 0.342 | 0.295 | A | B | B | B |
| IL-6 | 0.196 | 0.192 | 0.168 | 0.150 | A | A | A, B | B |
| IL-7 | 0.004 | 0.003 | 0.003 | 0.003 | A | B | B | B |
| IL-8 | 856.276 | 652.066 | 567.361 | 572.602 | A | B | B | B |
| MCP-1 | 0.053 | 0.047 | 0.039 | 0.039 | A | A, B | B | B |
| MDC | 0.399 | 0.407 | 0.345 | 0.339 | A | A | B | B |
| | Mean | | | | α = 0.05 | | | |
| | Baseline | Week 1 | Week 3 | Week 6 | Baseline | Week 1 | Week 3 | Week 6 |
| SAA | 7.039 | 6.905 | 6.092 | 5.162 | A | A | A, B | B |
| Tie-2 | 0.273 | 0.239 | 0.267 | 0.221 | A | A, B | A | B |
| VCAM-1 | 4.971 | 3.706 | 3.156 | 2.892 | A | B | B | B |
| VEGF | 0.625 | 0.511 | 0.478 | 0.480 | A | B | B | B |
| VEGF 2 | 0.772 | 0.661 | 0.620 | 0.582 | A | B | B | B |
| VEGF-D | 0.057 | 0.052 | 0.051 | 0.045 | A | A, B | A, B | B |
| VEGF-C | 0.145 | 0.149 | 0.125 | 0.137 | A, B | A | B | A, B |
| TARC | 0.020 | 0.029 | 0.019 | 0.019 | A | B | A | A |
| bFGF | 0.020 | 0.015 | 0.012 | 0.013 | A | A | A | A |
| Eotaxin-3 | 0.095 | 0.108 | 0.091 | 0.094 | A | A | A | A |
| Flt-1 | 0.390 | 0.518 | 0.433 | 0.415 | A | B | A, B | A |
| IL-12p40 | 0.039 | 0.031 | 0.028 | 0.031 | A | A | A | A |
| IL-2 | 0.166 | 0.199 | 0.210 | 0.162 | A | A | A | A |
| IL-8 (HA) | 47.508 | 44.362 | 41.260 | 39.119 | A | A | A | A |
| IP-10 | 0.540 | 1.688 | 0.740 | 0.606 | A | A | A | A |
| MCP-4 | 0.023 | 0.023 | 0.020 | 0.022 | A | A | A | A |
| MIP-1α | 0.091 | 0.091 | 0.084 | 0.080 | A | A | A | A |
| MIP-1β | 0.091 | 0.100 | 0.110 | 0.094 | A | A | A | A |
| TNFα | 2.009 | 2.067 | 2.021 | 1.670 | A | A | A | A |

### Example 16—One Hundred Seventy Metabolites were Identified in Gingival Samples

The same gingival-brush samples as described in EXAMPLE 15 were used for metabonomic analyses. Fourteen panelists were selected randomly from each treatment group to determine if any metabolite concentrations were changed in gingival samples during the first 3 weeks of treatment. Both baseline and week 3 samples were sent to Metabolon, Inc. (Durham, N.C.) for metabonomic measurement. 170 metabolites were identified and quantified. As shown in TABLE 7, some metabolite concentrations were changed during the first 3 weeks of treatment. Citrulline concentrations in the gingival samples were reduced after three weeks of treatment in the treatment regimen group. Similarly, ornithine was also reduced in the treatment regimen group after three weeks of treatment. Reduction of citrulline and ornithine was likely associated with alleviation of gingivitis.

TABLE 7
Comparison of metabolites in gingival brush samples between baseline and week 3
during gingivitis treatment

| | Baseline | 3 week | 3 week/ | | | |
|---|---|---|---|---|---|---|
| Biochemical Name | mean | mean | baseline | p-value | q-value | Mass |
| 13-HODE + 9-HODE | 1.0877 | 0.7088 | 0.65 | 0.0601 | 0.1338 | 295.2 |
| 1- | 1.2294 | 0.8274 | 0.67 | 0.038 | 0.1035 | 500.3 |
| arachidonoylglycerophosphoethanolamine | | | | | | |
| 1- | 0.7378 | 1.0747 | 1.46 | 0.0767 | 0.1548 | 478.3 |
| oleoylglycerophosphoethanolamine | | | | | | |
| 2-methylbutyrylcarnitine | 1.7769 | 0.6997 | 0.39 | 0.0034 | 0.0546 | 246.1 |
| (C5) | | | | | | |
| adenosine 5′- | 1.4092 | 0.8451 | 0.6 | 0.0295 | 0.0956 | 348.1 |
| monophosphate (AMP) | | | | | | |
| alanine | 0.8721 | 1.102 | 1.26 | 0.0318 | 0.0973 | 115.9 |
| arginylleucine | 1.4447 | 0.6819 | 0.47 | 0.0084 | 0.0777 | 288.3 |
| arginylphenylalanine | 0.9616 | 0.3335 | 0.35 | 0.0119 | 0.0777 | 322.2 |
| asparagylleucine | 0.9295 | 0.6122 | 0.66 | 0.0698 | 0.1465 | 246.2 |
| citrulline | 1.0147 | 0.71 | 0.7 | 0.0104 | 0.0777 | 176.1 |
| deoxycarnitine | 3.2381 | 0.6088 | 0.19 | 0.0003 | 0.0168 | 146.1 |
| EDTA | 1.5985 | 0.8384 | 0.52 | 0.0138 | 0.0777 | 291.1 |
| erythritol | 1.625 | 0.8085 | 0.5 | 0.0582 | 0.1325 | 217 |
| fructose | 1.9933 | 1.1106 | 0.56 | 0.0847 | 0.1605 | 217 |
| glutamine | 1.2459 | 0.8366 | 0.67 | 0.0374 | 0.1035 | 147.2 |
| glutathione, oxidized | 1.0161 | 1.4669 | 1.44 | 0.087 | 0.1605 | 613.1 |
| (GSSG) | | | | | | |
| glycerol | 1.3783 | 0.8308 | 0.6 | 0.0391 | 0.1035 | 205 |
| lauryl sulfate | 1.685 | 0.8623 | 0.51 | 0.0397 | 0.1035 | 265.2 |
| leucine | 1.2158 | 0.9359 | 0.77 | 0.0613 | 0.1338 | 132.2 |
| leucylleucine | 0.9505 | 0.4393 | 0.46 | 0.0251 | 0.0877 | 245.1 |
| lysylleucine | 1.2009 | 0.5275 | 0.44 | 0.0036 | 0.0546 | 260.2 |
| lysylphenylalanine | 1.1682 | 0.4563 | 0.39 | 0.0095 | 0.0777 | 294.3 |
| maltose | 0.8727 | 1.4481 | 1.66 | 0.022 | 0.0877 | 204.1 |
| maltotriose | 1.0456 | 1.8347 | 1.75 | 0.0858 | 0.1605 | 204 |
| mannitol | 1.3004 | 0.7982 | 0.61 | 0.042 | 0.107 | 319.1 |
| ornithine | 1.2916 | 0.7069 | 0.55 | 0.0367 | 0.1035 | 141.9 |
| palatinitol | 1.4395 | 0.8272 | 0.57 | 0.0782 | 0.1549 | 204 |
| phosphate | 1.4008 | 0.8376 | 0.6 | 0.0208 | 0.0877 | 298.9 |
| proline | 1.405 | 0.99 | 0.7 | 0.0033 | 0.0546 | 116.1 |
| propionylcarnitine | 1.2565 | 0.7688 | 0.61 | 0.0201 | 0.0877 | 218.2 |
| pyroglutamine | 1.3424 | 0.7873 | 0.59 | 0.0136 | 0.0777 | 129.2 |
| serylisoleucine | 1.1753 | 0.7169 | 0.61 | 0.0814 | 0.1583 | 219.2 |
| spermidine | 1.1613 | 0.8678 | 0.75 | 0.0687 | 0.1465 | 146.2 |
| succinate | 1.2929 | 0.8113 | 0.63 | 0.0754 | 0.1548 | 247 |
| threonylleucine | 1.1513 | 0.4931 | 0.43 | 0.0044 | 0.0594 | 231.2 |
| threonylphenylalanine | 1.7693 | 0.918 | 0.52 | 0.0233 | 0.0877 | 267.2 |
| trehalose | 2.3563 | 0.9084 | 0.39 | 0.0054 | 0.0647 | 361.2 |
| tryptophan | 1.1518 | 0.9089 | 0.79 | 0.0487 | 0.1185 | 205.1 |
| tyrosine | 1.383 | 1.0299 | 0.74 | 0.0161 | 0.0787 | 182.1 |
| valine | 1.1598 | 0.9271 | 0.8 | 0.0304 | 0.0956 | 118.1 |
| valylvaline | 0.9347 | 0.8231 | 0.88 | 0.0508 | 0.1207 | 215.2 |
| X-13671 | 0.5035 | 0.918 | 1.82 | 0.0545 | 0.1267 | 315.3 |
| X-14588 | 1.3647 | 0.8378 | 0.61 | 0.024 | 0.0877 | 151 |
| X-16103 | 1.3643 | 0.8461 | 0.62 | 0.0297 | 0.0956 | 99.3 |
| X-17266 | 1.3158 | 0.576 | 0.44 | 0.0003 | 0.0168 | 530.4 |
| X-17375 | 1.4785 | 0.8387 | 0.57 | 0.0189 | 0.0877 | 357.1 |
| X-18472 | 0.6138 | 1.1441 | 1.86 | 0.0011 | 0.0405 | 827.1 |
| X-18779 | 1.3756 | 0.8035 | 0.58 | 0.0162 | 0.0787 | 209.1 |
| X-19607 | 1.5237 | 0.7167 | 0.47 | 0.002 | 0.0537 | 366.1 |
| X-19609 | 1.3284 | 0.7721 | 0.58 | 0.016 | 0.0787 | 204 |
| X-19612 | 1.3896 | 0.7843 | 0.56 | 0.01 | 0.0777 | 427.2 |
| X-19613 | 1.3412 | 0.7535 | 0.56 | 0.0099 | 0.0777 | 429.3 |
| X-19614 | 1.3378 | 0.7343 | 0.55 | 0.0454 | 0.113 | 570.1 |
| X-19807 | 1.3478 | 0.8411 | 0.62 | 0.0244 | 0.0877 | 93 |
| X-19808 | 1.3348 | 0.8368 | 0.63 | 0.0254 | 0.0877 | 95 |
| X-19850 | 1.3576 | 0.7519 | 0.55 | 0.011 | 0.0777 | 334.2 |
| X-19857 | 1.3357 | 0.8032 | 0.6 | 0.038 | 0.1035 | 230 |
| X-20000 | 1.2784 | 0.7536 | 0.59 | 0.0133 | 0.0777 | 81.2 |

### Example 17—Citrulline was Reduced in Regimen Treatment Over 6 Weeks

Quantitation of citrulline and ornithine from the extracts of the Gingival-brush samples was conducted using gradient hydrophilic interaction liquid chromatography with tandem mass spectrometry (HILIC/MS/MS). Gingival-brush samples were obtained from the same human panelists in the clinical study as described in EXAMPLE 14, and were placed into extraction buffer as described in EXAMPLE 15. The supernatants were subject to both HILIC/MS/MS and BCA analysis. For free citrulline and ornithine analysis, the extracts of the Gingival-brush samples were analyzed either directly (50 μl undiluted sample solution) in 50/50 acetonitrile/ultra-pure water with 0.754% formic acid or diluted fivefold. For total citrulline and ornithine analysis, the extracts of the Gingival-brush samples were first hydrolyzed using 6 N HCl (50 μL of extract with 450 μL of 6N HCl), no shaking, and placed on a hot plate at 110° C. for 16 hours. The hydrolyzed samples were then dried down under vacuum at room temperature (Savant speedvac of Lifetechnology, Grand Island, N.Y.) and then reconstituted in 1 ml of dilution solution (50/50 acetonitrile/ultra-pure water with 0.754% formic acid) for analysis. The standards and the samples were analyzed using gradient hydrophilic interaction liquid chromatography with tandem mass spectrometry (HILIC/MS/MS). Analytes and the corresponding ISTDs (stable isotope labeled internal standard) were monitored by electrospray ionization (ESI) in positive mode using the selected-reaction-monitoring schemes shown in TABLE 8. A standard curve was constructed by plotting the signal, defined here as the peak area ratio (peak area analyte/peak area ISTD), for each standard versus the mass of each analyte for the corresponding standard. The mass of each analyte in the calibration standards and Gingival-brush extract samples were then back-calculated using the generated regression equation. The concentration of protein bound citrulline or ornithine was calculated as the result of subtracting the concentration of free citrulline or ornithine from the concentration of total citrulline or ornithine, respectively. The result was reported as the concentration of citrulline or ornithine or the result was standardized by dividing by the amount of citrulline or ornithine by the amount of the total proteins that were found in the extract.

TABLE 8
Multiple Reaction Monitoring (MRM) transitions
for analytes and their corresponding stable
isotope labeled internal standards

| Analytes | MRM | Internal Standards | MRM |
|---|---|---|---|
| Citrulline | 176 → 159 | d₇-Citrulline | 181 → 164 |
| Ornithine | 133 → 70 | d₆-Ornithine | 139 → 76 |

All samples from all panelists of the Test regimen [Crest® Pro-Health Clinical Plaque Control (0.454% stannous fluoride) dentifrice, Oral-B® Professional Care 1000 with Precision Clean brush head and Crest® Pro-Health Refreshing Clean Mint (0.07% CPC) mouth rinse] were analyzed. As shown in FIG. 16, citrulline levels reduced rapidly in the first week of treatment, and then continued to decline gradually in weeks 3 and 6 of treatment. These results are consistent with clinical observations, where gingival bleeding sites (GBI) and the gingival inflammation (MGI) were reduced over the 6-week treatment period.

### Example 18—Levels of Proteins Containing Ornithine Decreased Over 6 Week Treatment

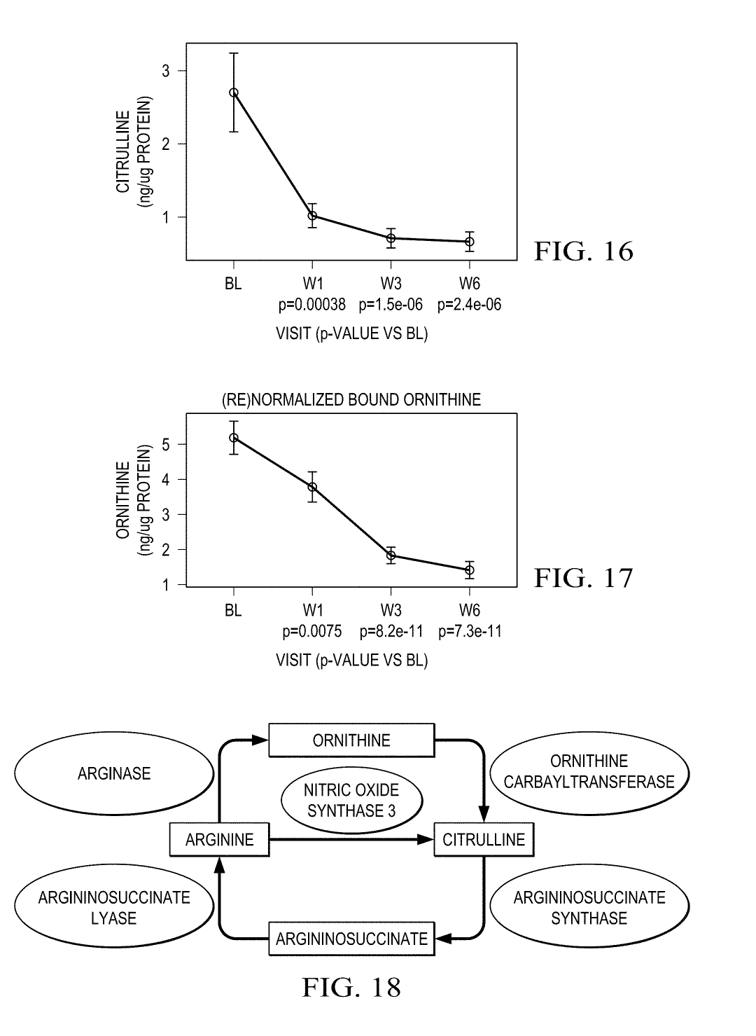

The same samples as described in EXAMPLE 17 were analyzed using procedures as described in EXAMPLE 17. Gingivitis was treated for 6 weeks. Baseline (BL) represents diseased status. Symptoms of gingivitis were alleviated from week 1 to week 6 treatments. Protein bound ornithine (the difference between total and the free ornithine) was higher in gingivitis as shown in FIG. 17. Protein bound ornithine was reduced gradually as gingivitis was decreased in severity.

### Example 19—Expression of Enzymes in the Ornithine-Citrulline-Arginine Cycle was Changed in Gingival Samples During the 6 Week Treatment

Gingival samples were collected as described in EXAMPLES 15, from the same panelists as in EXAMPLE 15, and were used to examine the expression of genes during 6 weeks of treatments with Test regimen [Crest® Pro-Health Clinical Plaque Control (0.454% stannous fluoride) dentifrice, Oral-B® Professional Care 1000 with Precision Clean brush head and Crest® Pro-Health Refreshing Clean Mint (0.07% CPC) mouth rinse] and Control regimen [Crest® Cavity Protection (0.243% sodium fluoride) dentifrice and Oral-B® Indicator Soft Manual toothbrush].

After harvesting the samples, the brush was completely immersed in the RNAlater solution (1 ml in a 1.5 ml Eppendorf tube) for keeping RNA from degrading during transport and storage (Qiagen, Valencia, Calif.). The microcentrifuge tubes were vortexed/mixed for 30 seconds, immediately frozen on dry ice, stored and transferred on dry ice to the lab for biomarker analysis. RNA isolation and microarray analysis were performed as described previously in a publication (Offenbacher S, Barros S P, Paquette D W, Winston J L, Biesbrock A R, Thomason R G, Gibb R D, Fulmer A W, Tiesman J P, Juhlin K D, Wang S L, Reichling T D, Chen K S, Ho B. J Periodontol. 2009 December; 80(12):1963-82. doi: 10.1902/jop.2009.080645. Gingival transcriptome patterns during induction and resolution of experimental gingivitis in humans).

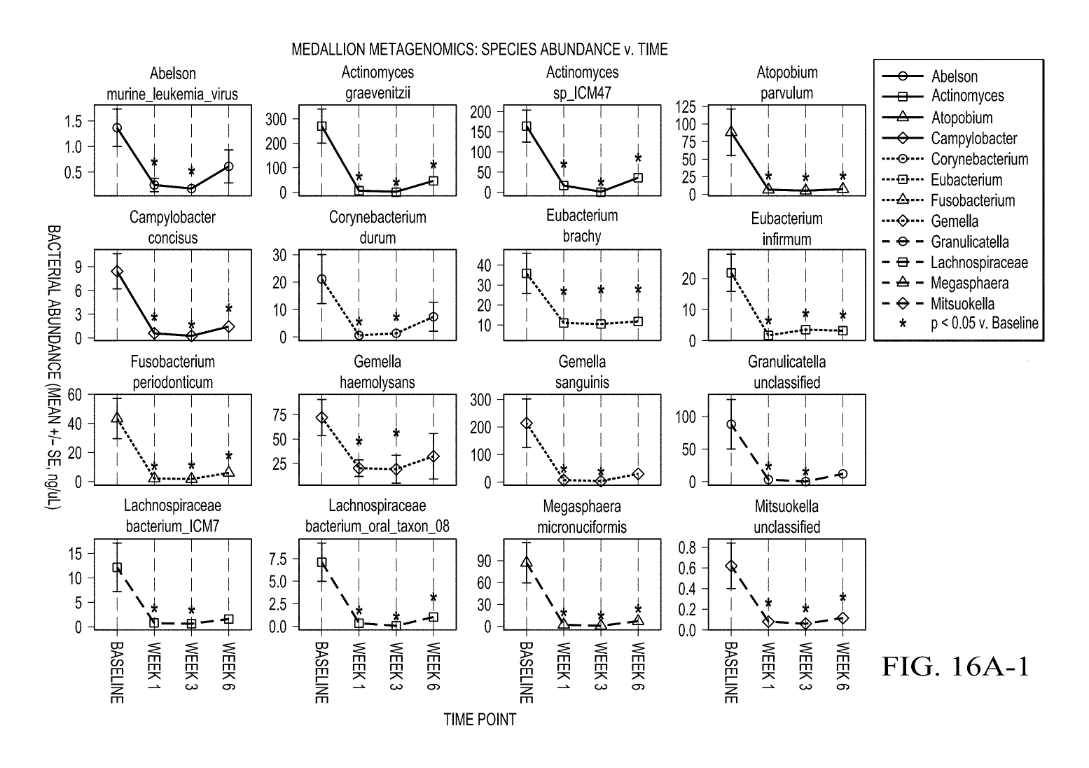

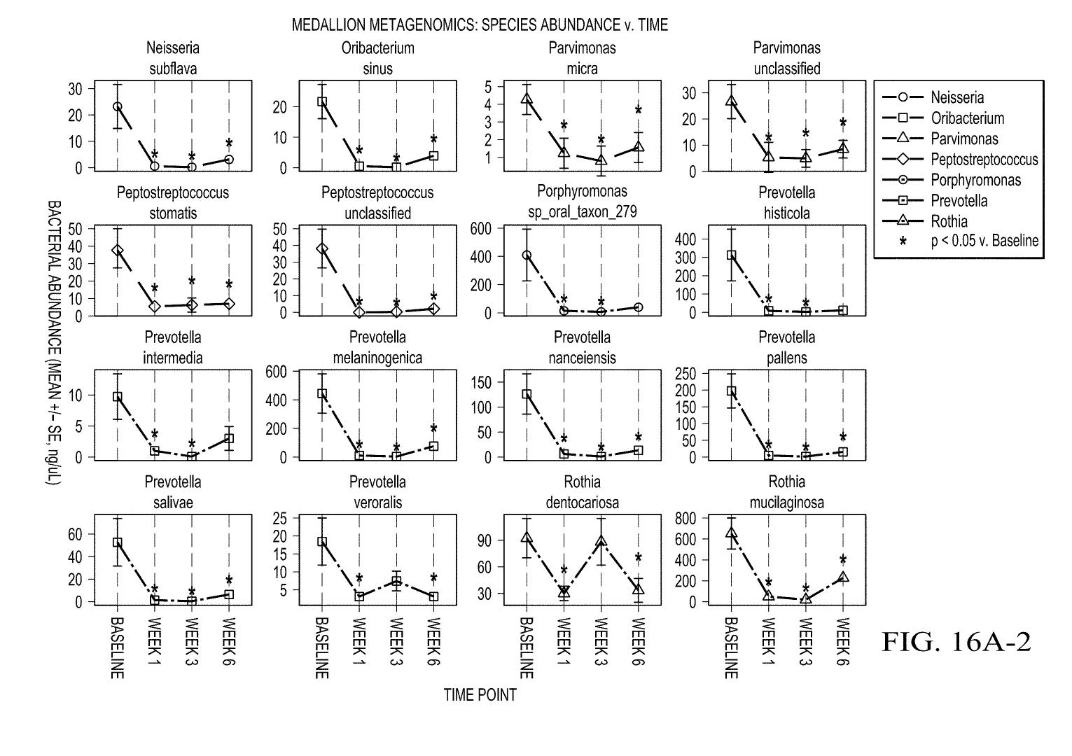

The ornithine-citrulline-arginine cycle consists of four enzymes (FIG. 18). The main feature of the cycle is that three amino acids (arginine, ornithine, and citrulline) can be converted to each other. The first enzyme is ornithine transcarbamoylase, which transfers a carbamoyl group from carbamoyl phosphate to ornithine to generate citrulline. This reaction occurs in the matrix of the mitochondria. Expression of ornithine transcarbamoylase was reduced in the treatment (FIG. 19). The second enzyme is argininosuccinate synthetase. This enzyme uses ATP to activate citrulline by forming a citrullyl-AMP intermediate, which is attacked by the amino group of an aspartate residue to generate argininosuccinate. This and subsequent two reactions occur in the cytosol. Again, expression of argininosuccinate synthetase decreased during the treatment. The third enzyme is argininosuccinate lyase, which catalyzes cleavage of argininosuccinate into fumarate and arginine. The last enzyme is argininase. Argininases cleave arginine to produce urea and ornithine. In a contrast to the decreased expression of ornithine transcarbamoylase and argininosuccinate synthetase genes, argininase I and II increased (FIG. 19).

Arginine is also a substrate for nitric oxide synthase, which oxidizes arginine to produce citrulline and nitric oxide. Expression of nitric oxide synthase gene increased too (FIG. 19).

### Example 20—Citrulline Increased in Gingival Samples in Experiment Gingivitis

Experimental gingivitis: Another clinical study was carried out to determine whether citrulline is increased in experimentally induced gingivitis in healthy human panelists. This was a case-control study enrolling 60 panelists. The study population included two groups as follows: Group 1 or high bleeders group, thirty (30) panelists with at least 20 bleeding sites, where bleeding is a GBI site score of 1 or 2 at baseline. Group 2 or low bleeders group, thirty (30) panelists with 2 or less bleeding sites, where bleeding is a GBI site score of 1 or 2.

The study consisted of two Phases: Health/Rigorous Hygiene Phase with dental prophylaxis, polishing and rigorous oral hygiene; and Induced Gingivitis Phase without oral hygiene. At the Screening visit, panelists underwent an oral soft tissue assessment and had a gingivitis evaluation (Modified Gingival Index (MGI) and Gingival Bleeding Index (GBI). At Visit 2 qualifying panelists received an oral soft tissue exam followed by a gingivitis evaluation and gingival plaques and gum swabs were collected for the qPCR, protein and RNA host biomarker analysis. Following that, all panelists received dental prophylaxis and entered the Health/Rigorous Hygiene Phase, lasting two weeks. After two weeks of rigorous hygiene, panelists entered the Induced Gingivitis Phase, lasting for three weeks. Oral soft tissue exams and gingivitis were re-evaluated and all samples (gum swabs) were collected at Baseline, WK0 and WK2.

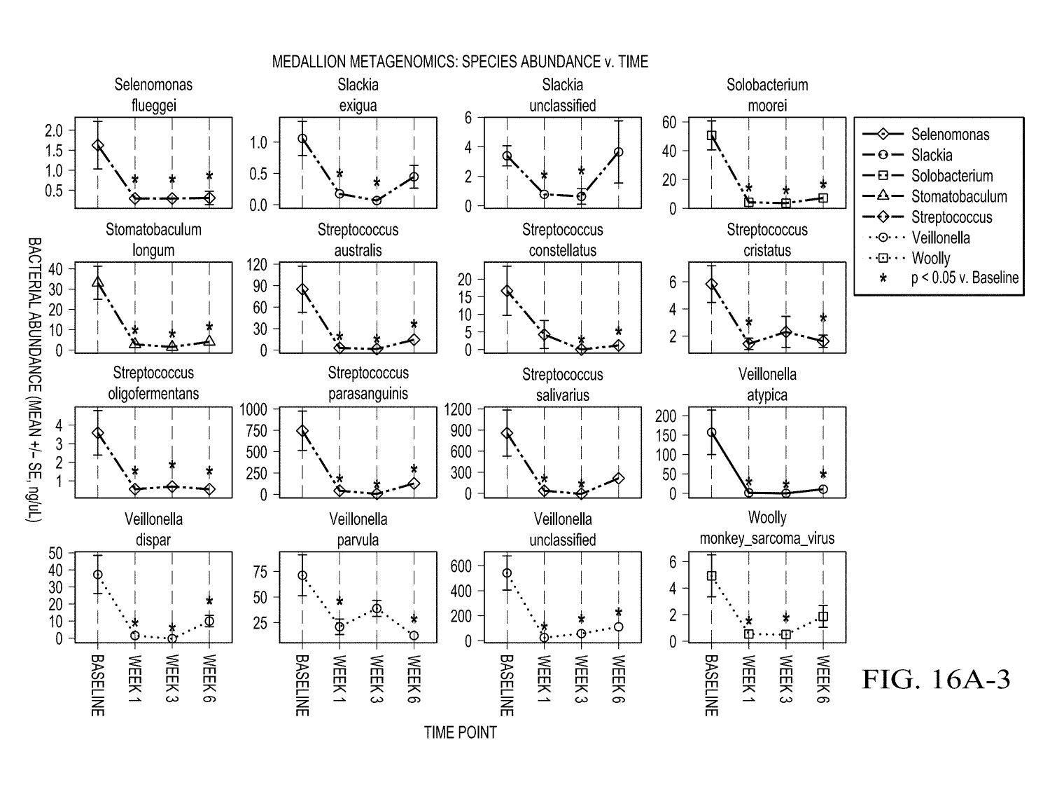

Gingival sample collection—A gum swab was collected from each side of the upper arch using the procedures as described in EXAMPLE 15. Gum swabs were collected close to the gum line from the buccal sites only (preferably from four adjacent teeth—preferably from premolar and molar areas). Panelists rinsed for 30 seconds with 15 ml of Listerine rinse to clean the surface of sampling area. After the Listerine rinse, panelists rinsed for 30 seconds with 20 ml of water. Following that, selected sites were isolated with a cotton roll and gently dried with an air syringe and two gum swabs were taken with collection brushes/swabs from the gingiva region close to the gumline of the selected teeth. The samples were placed in a pre-labeled (panelist ID, sample ID, visit, and date) 1.5 ml micro-centrifuge tube containing 800 ul DPBS (Dulbecco's phosphate-buffered saline) (Lifetechnologies, Grand Island, N.Y.) with protease inhibitors, including AEBSF (4-(2-Aminoethyl) benzenesulfonyl fluoride hydrochloride) 2 mM, aprotinin 0.3 μM, Bestatin 130 μM, EDTA (Ethylenediaminetetraacetic acid) 1 mM, E-64 1 μM, and leupeptin 1 μM. The vials were vortexed/mixed for 30 seconds, immediately frozen on dry ice, stored and transferred on dry ice to the lab for biomarker analysis. Samples from three visits were analyzed using the procedures described in EXAMPLE 17, and shown in FIG. 20. Those three visits were baseline, Week 0, (right after the Health/Rigorous Hygiene Phase and before the induced gingivitis phase) and week 2 (at the end of Induced Gingivitis Phase). Free citrulline levels were low in both the high and low bleeders groups at the baseline and week 0, but rose quickly in the induced gingivitis in both groups at week 2.

### Example 21—Levels of Proteins Containing Citrulline Decreased in Experimentally Induced Gingivitis

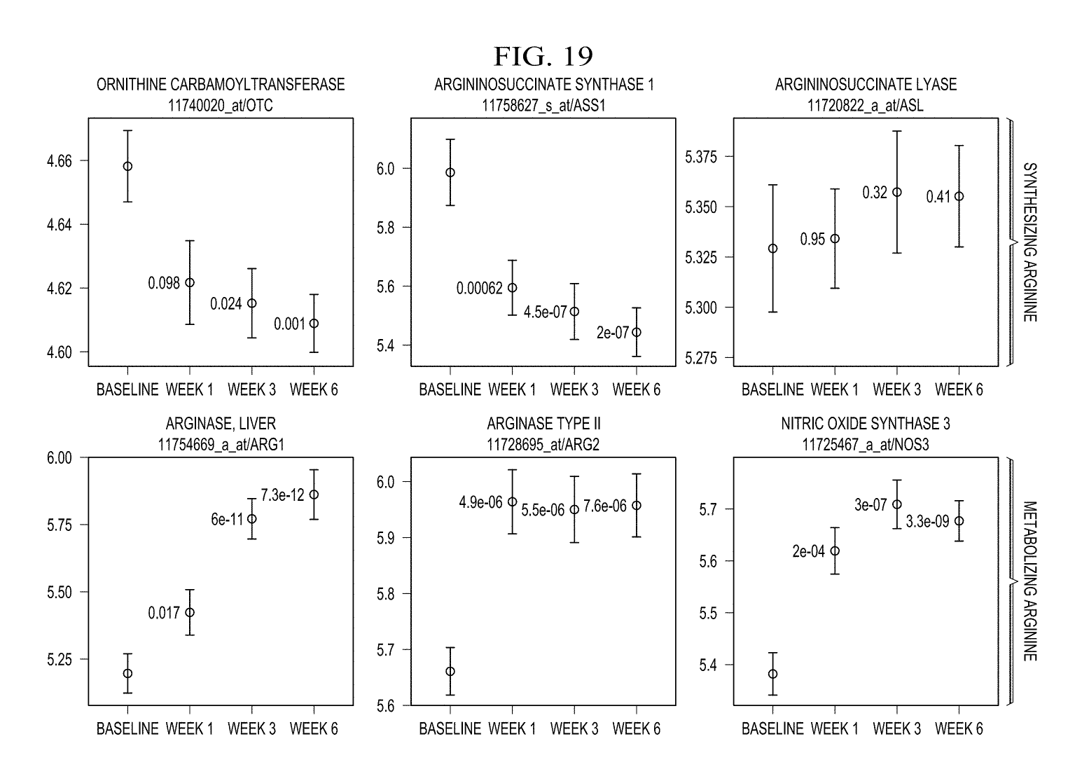

The same procedures were used as described in EXAMPLE 17. The samples were the same as described in EXAMPLE 20. Protein bound citrulline was lower at the baseline than that at week 0 in both high and low bleeders groups as shown in FIG. 21 in gingival tissue. It was low in experimental gingivitis in both groups at week 2.

### Example 22—Levels of Proteins Containing Ornithine Increased in Gingival Samples in Experiment Gingivitis

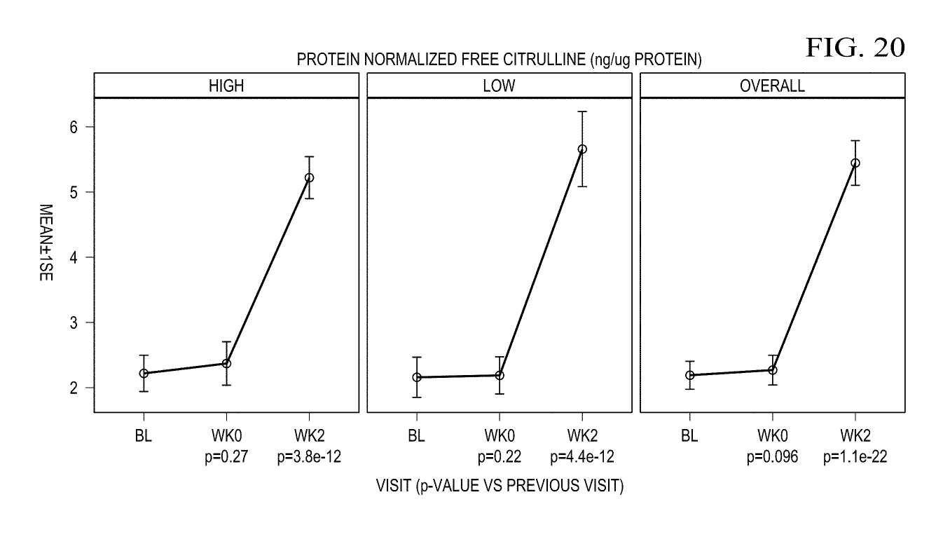

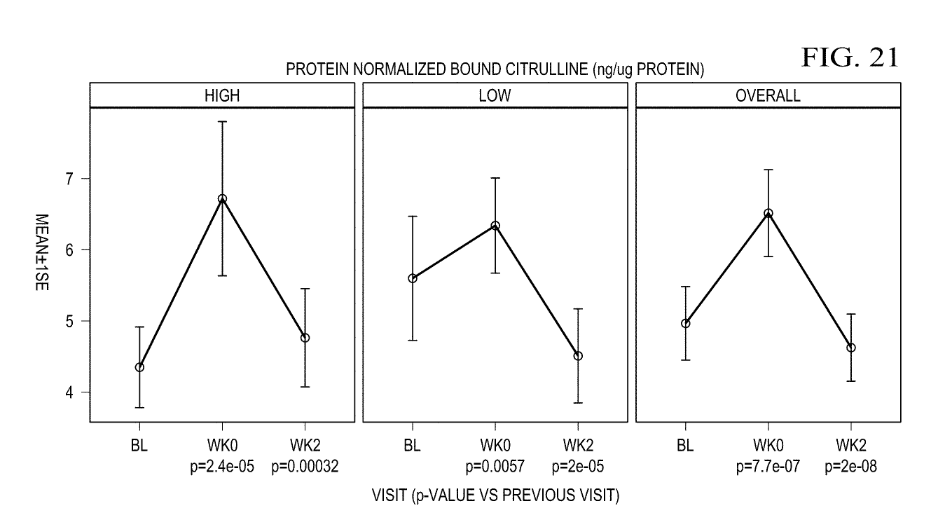

The same clinical samples from experimental gingivitis (EXAMPLE 20) were analyzed using the procedures described in EXAMPLE 17. The bound ornithine was the lowest at week 0 (FIG. 22) in both groups. Its levels at the baseline were higher than those at week 0. The bound ornithine reached peaks when gingivitis was induced in both groups at week 2. Also it is worth noting the total ornithine (Free and protein bound ornithine) was increased in the induced gingivitis (FIG. 23) in both groups.

### Example 23—Levels of Proteins Containing Arginine Decreased in Gingival Samples in Experimentally Induced Gingivitis

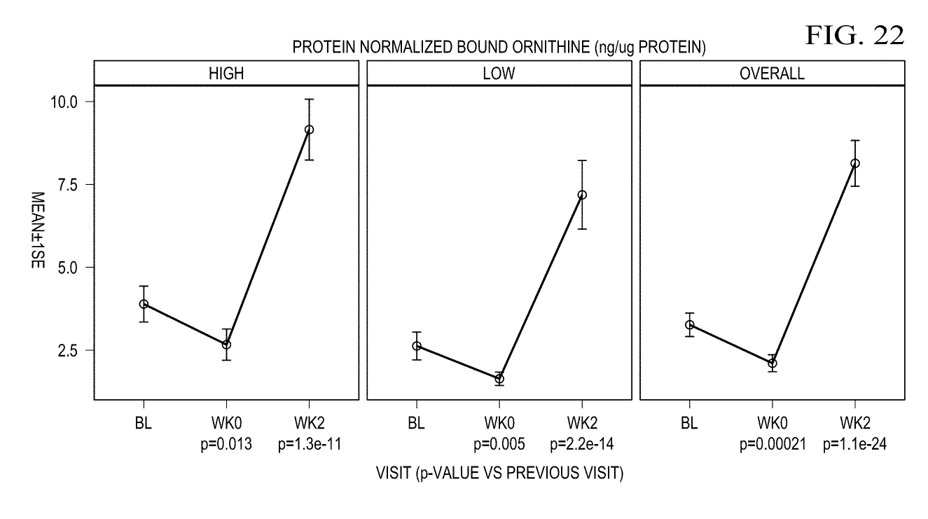

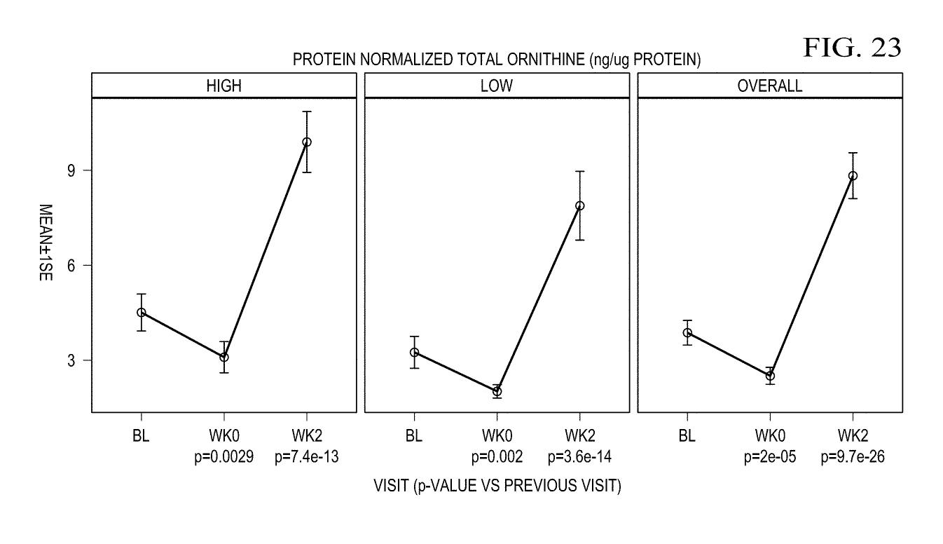

The same procedures were used as described in EXAMPLE 17. The samples were the same as described in EXAMPLE 20. The protein bound arginine was the lowest in induced gingivitis (FIG. 24) in both groups. Its levels were higher in WK0 than at Baseline in both groups. The total arginine in the gingival brush samples displayed the same patterns as the protein bound one (FIG. 25).

### Example 24—Citrulline Inhibited LPS Stimulated Production of Proinflammatory Cytokine in Human THP-1 Cells

Citrulline was purchased from Sigma-Aldrich (St. Louis, Mo.). THP1-Dual™ cells were purchased from Invivogen (San Diego, Calif.). Cells were cultured following the manufacturer's instruction, as described in EXAMPLE 11. For treatment, 0.3 mM to 9 mM of citrulline were first added to the culture medium. Then, 300 ng/ml of P. gingivalis LPS 1690 were added 60 minutes later. After 24 hours of treatment, media was collected and analyzed for cytokine production using 9-plex kit (Meso Scale Diagnostics Rockville, Md.).

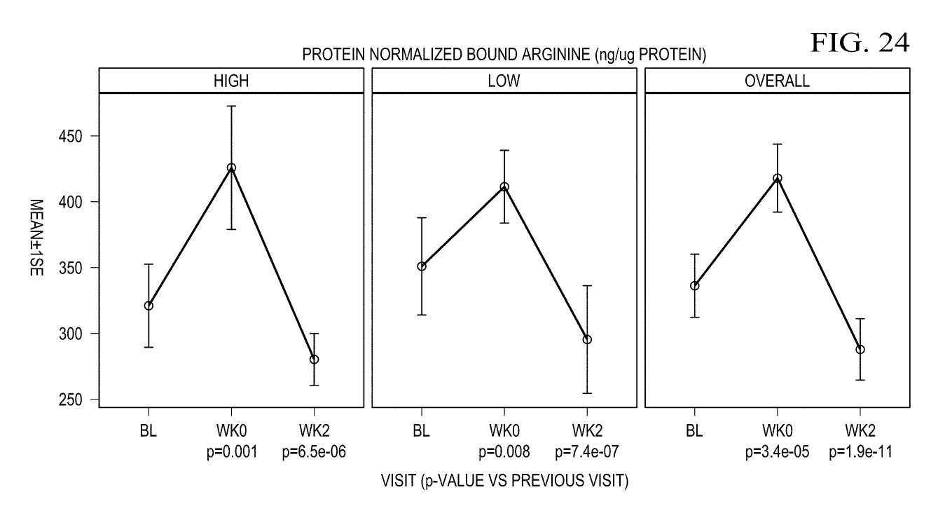

P. gingivalis LPS 1690 stimulated cytokine production, as shown in FIG. 26. Citrulline inhibited P. gingivalis LPS 1690 effects on proinflammatory cytokine production in a dose-dependent manner. Those cytokines include IL-6, TNF-α, IL-12p70, IL-10, IL-2, IFN-r and IL-1β.

### Example 25—Binding of BODIPY-TR-Cadaverine to Bacteria and their Products

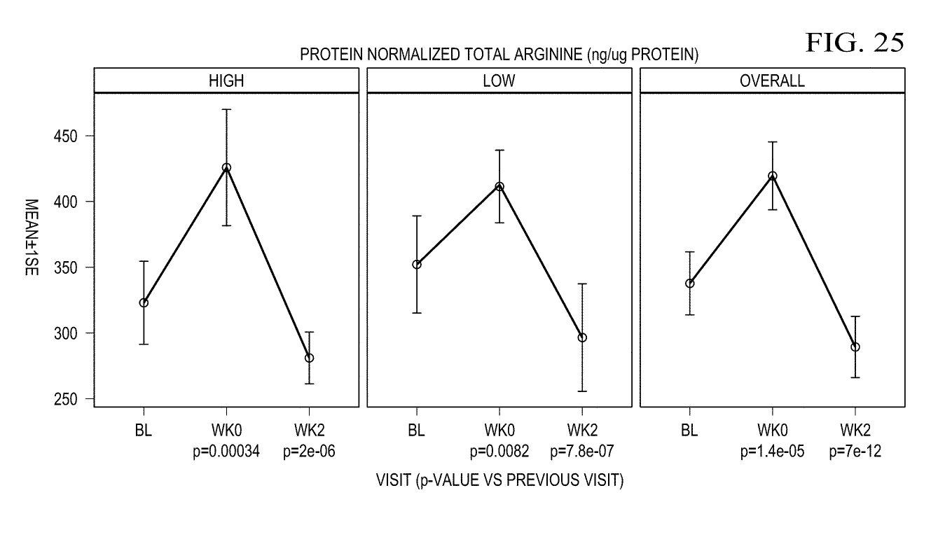

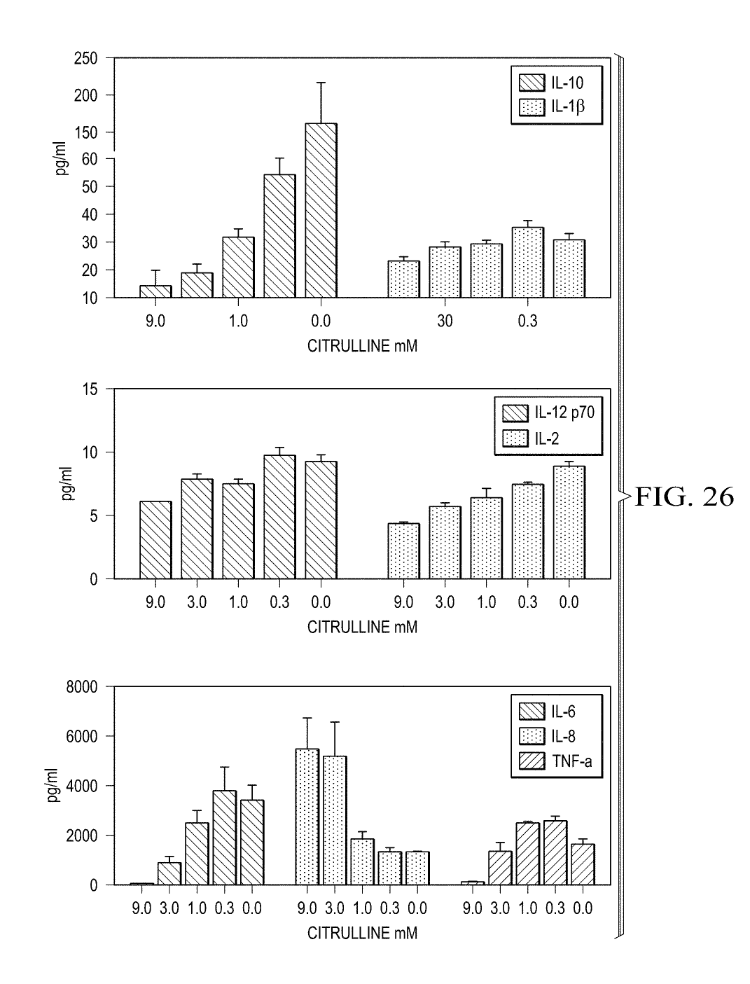

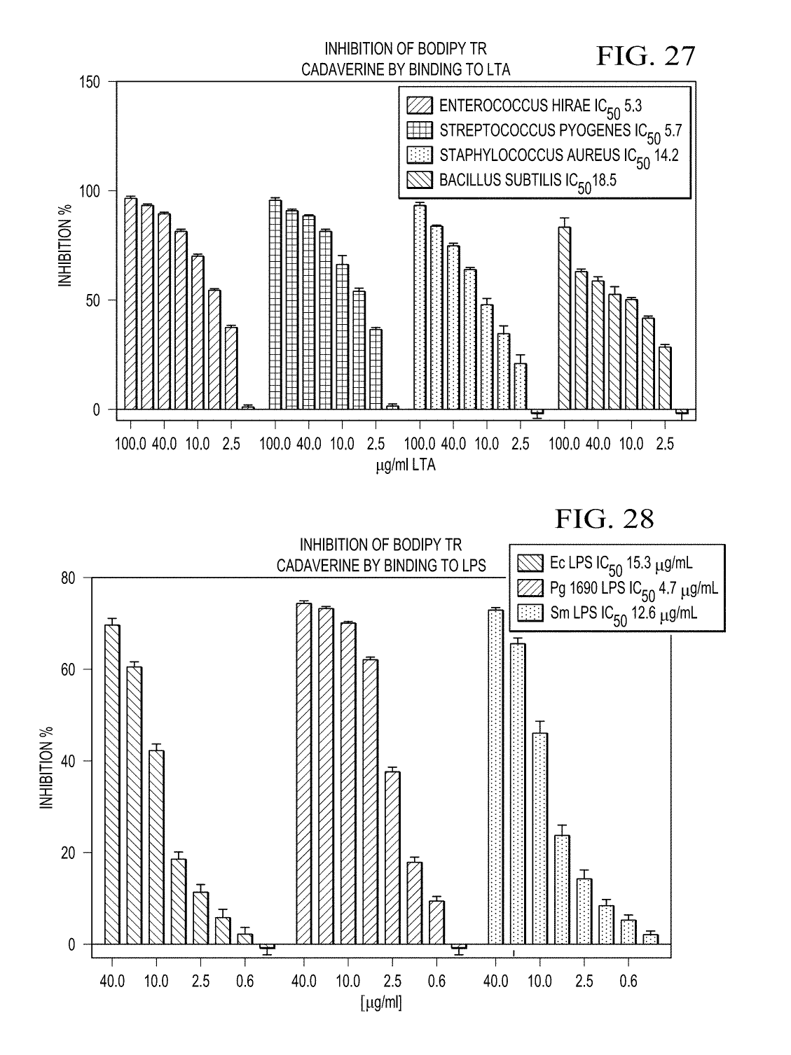

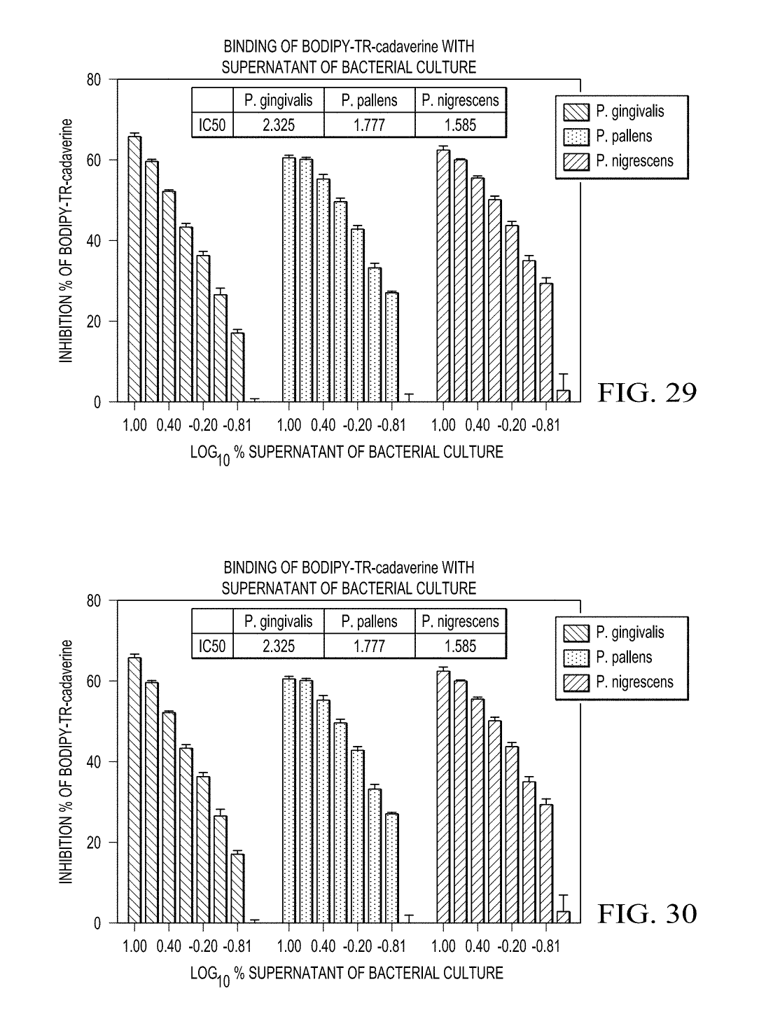

Bacteria and their products can activate TLR2 and TLR4 reporter genes as described in EXAMPLES 5 and 8. Here, experiments were carried out to determine whether different bacteria and their products have different IC50 in reducing fluorescent intensity of BODIPY-TR-cadaverine, hereinafter referred to as BC, in a procedure as described in EXAMPLE 4. Briefly, high throughput screen utilizes the fluorescent dye BODIPY-TR-cadaverine 5-(((4-(4,4-difluoro--(2-thienyl)-4-bora-3a,4a-diaza-s-indacene-3-yl)phenoxy)acetyl)amino)pentylamine hydrochloride (BC fluorescent dye), purchased from Life Technologies (Carlsbad, Calif.), as described previously by Wood, Miller and David (2004) (Comb Chem High Throughput Screen 2004 May; 7(3):239-49. Anti-endotoxin agents. 1. Development of a fluorescent probe displacement method optimized for the rapid identification of lipopolysaccharide-binding agents.). The experiment was carried out at room temperature. E. coli LPS (15 μg/ml) was mixed in a well of a 96-well solid black flat bottom plate (Corning Incorporated, Corning, N.Y.) with SnF2 and cetylpyridinium chloride in 30 μl of 50 mM Tris buffer at pH 7.4 for 10 min, and then 20 μl of 60 μM BC fluorescent dye was added right before fluorescence measurement at 21° C., or room temperature. Fluorescent measurements were performed in a SpectraMax M3 automated 96 well plate reader (Molecular Device, Sunnyvale, Calif.). The excitation wavelength was 580 nM and the fluorescence emission was measured at 620 nM. The half maximal inhibitory concentration (IC₅₀) is a measure of the effectiveness of bacterial products inhibiting BC emitting fluorescence. It indicates how much of a bacterial material is needed to inhibit BC fluorescence by half using GraphPad Prism software (GraphPad Software, La Jolla, Calif.). As shown in FIGS. 27 and 28, each bacterial product decreases the fluorescent intensity of BC in a dose-dependent manner. Similarly, the whole bacterial cells and the supernatant of bacterial growth also reduced the fluorescence of BC in a dose-dependent fashion (FIGS. 29 and 30).

### Example 26—Determination of Bacterial Virulence

Growth of bacteria: Two bacteria, Bacterium A and Bacterium B, were cultured in Tryptic Soy Broth medium (Sigma-Aldrich, St. Louis, Mo.) at 37° C. with shaking at 200 rpm. The bacteria were harvested at 24 hours, and suspended in 0.5 ml of phosphate-buffered saline, labeled “live”. Half ml of “Live” bacteria was transferred to a 1.5 ml microtube, and heated to 80° C. for 30 min. The heat-treated bacteria were labeled “Heat-Inactivated”, or “Dead”.

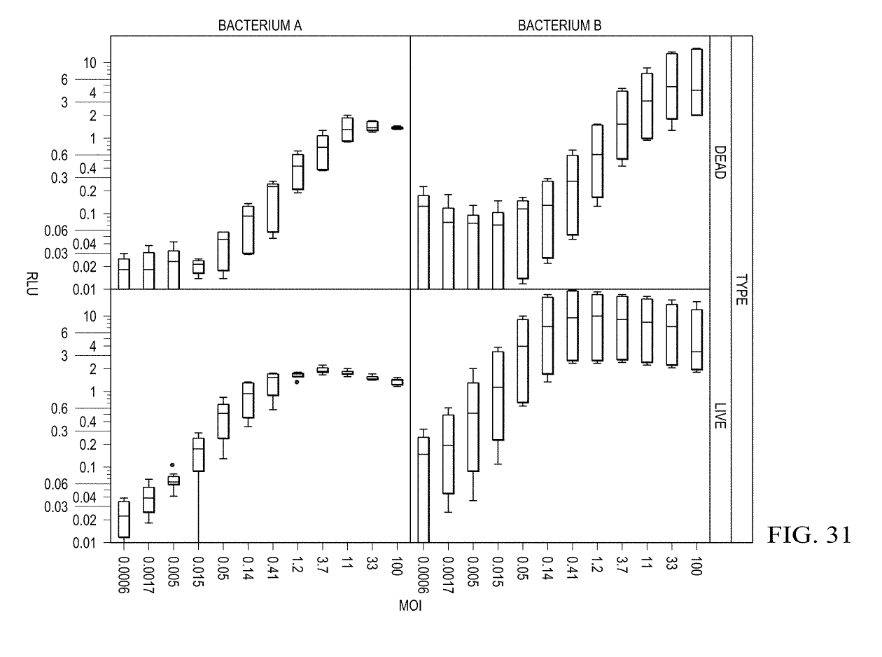

Measurement of TLR responses in THP-1 gene reporter cells (NFkB-SEAP): The Live and Heat-Inactivated bacteria were applied to THP-1 cells as described in EXAMPLE 11. As shown in FIG. 31, EC50 of Bacterium A and B on activation of NFkB-SEAP reporter gene in THP-1 cells was determined. Both Live and Heat-inactivated (Dead) bacteria stimulated expression of the NFkB-SEAP reporter gene. Bacterium B had a lower EC50 than Bacterium A in activating expression of the NFkB-SEAP reporter gene.

Cytokine production and measurement: Human peripheral bleed mononuclear cells (hPBMC) were obtained from All Cells company (All Cells, Alameda, Calif.) as Leukapheresed blood. Leukapheresed blood was mixed with an equal part of DMEM+glutaGRO supplemented with 9.1% fetal bovine serum and 1% penicillin/streptomycin (Thermo Fisher, Waltham, Mass.). hPBMC were isolated from the 1:1 mixture of blood and culture medium by collecting the buffy coat of a centrifuged Histopaque®-1077 (Sigma-Aldrich, St. Louis, Mo.) buffer density gradient. The cells (200,000 cells) were cultured in 200 μl of DMEM+glutaGRO supplemented with 9.1% fetal bovine serum and 1% penicillin/streptomycin, and treated with Live and Heat-Inactive bacteria (6,250,000 colony-forming units). The medium was harvested at 24 hours after adding the bacteria, and analyzed for proinflammatory cytokines in a kit following manufacturer's instruction (Meso Scale Diagnostics, Rockville, Md.).

As shown in TABLE 9, both live bacterium A and B stimulated production of cytokines in hPBMC. Bacteriun B was far more potent than Bacterium A in promoting production of IFN-γ, IL-10, IL-12p70, IL-1β, IL-6, IL-8 and TNF-α in hPBMC.

| | | Live | Live |
|---|---|---|---|
| Statistics | Cytokines | Bacterium A | Bacterium |
| Mean | IFN-γ | 867.16 | 12734.36 |
| Mean | IL-10 | 68.35 | 412.51 |
| Mean | IL-12p70 | 23.75 | 253.47 |
| Mean | IL-1β | 2300.86 | 6969.31 |
| Mean | IL-2 | 44.24 | 65.17 |
| Mean | IL-6 | 3431.05 | 9963.84 |
| Mean | IL-8 | 65742.91 | 70357.25 |
| Mean | TNF-α | 3710.66 | 13825.49 |
| Std Dev | IFN-γ | 488.11 | 8200.13 |
| Std Dev | IL-10 | 33.56 | 314.23 |
| Std Dev | IL-12p70 | 14.71 | 283.33 |
| Std Dev | IL-1β | 1569.72 | 7691.93 |
| Std Dev | IL-2 | 32.97 | 36.29 |
| Std Dev | IL-6 | 2212.62 | 6552.12 |
| Std Dev | IL-8 | 18689.24 | 13669.28 |
| Std Dev | TNF-α | 2503.25 | 8302.26 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of determining bacterial virulence in an oral cavity comprising:
a. providing a reporter cell expressing at least one toll-like receptor;
b. providing a sample of oral matter;
c. combining the sample of oral matter and the reporter cell;
d. measuring the toll-like receptor activation.

2. The method of claim 1, wherein the toll-like receptor is at least one of TLR1 to TLR9.

3. The method of claim 1, wherein the reporter cell expresses at least of one TLR2 or TLR4.

4. The method of claim 1, wherein the toll-like receptor activates a reporter gene.

5. The method of claim 4, wherein the reporter gene is a secreted embryonic alkaline phosphatase (SEAP) reporter gene.

6. The method of claim 1, wherein the sample of oral matter is at least one of gum-line plaque, subgingival plaque, supragingival plaque, interstitial plaque, gingival crevicular fluid (GCF), gingival biopsy, saliva, or tongue swab.

7. The method of claim 1, wherein the sample of oral matter comprises virulence factor that is at least one of Gram positive cell wall components, Gram negative cell wall components, bacterial DNA, flagellin, peptidoglycan, bacterial metabolites, fimbriae; outer membrane vesicles bacterial proteins, or bacterial enzymes.

8. The method of claim 7, wherein the sample of oral matter comprises virulence factor that is at least one of lipopolysaccharide or lipoteichoic acid.

9. The method of claim 1 comprising the additional steps of:
e. providing another sample of oral matter from an individual who used an oral care composition prior to providing another sample of oral matter;
f. combining the another sample of oral matter and the reporter cell;
g. measuring the toll-like receptor response;
h. comparing the toll-like receptor response from the sample of oral matter and another sample of oral matter.

10. The method of claim 9, where the sample of oral matter


SEQUENCE LISTING (9 sequences)

SEQ ID NO: 1 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 2 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 3 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 4 - protein, 798 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATS

SEQ ID NO: 5 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 6 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGPTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 7 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 8 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 9 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE